SEQUENCE LISTING (2 sequences)

SEQ ID NO: 1 - protein, 191 aa, Homo sapiens [sequence omitted]

SEQ ID NO: 2 - protein, 638 aa, Homo sapiens
MDLWQLLLTLALAGSSDAFSGSEATAAILSRAPWSLQSVNPGLKTNSSKEPKFTKCRSPERETFSCHWTDEVHHGTKNLGPIQLFYTRRNTQEWTQEWKECPDYVSAGENSCYFNSSFTSIWIPYCIKLTSNGGTVDEKCFSVDEIVQPDPPIALNWTLLNVSLTGIHADIQVRWEAPRNADIQKGWMVLEYELQYKEVNETKWKMMDPILTTSVPVYSLKVDKEYEVRVRSKQRNSGNYGEFSEVLYVTLPQMSQFTCEEDFYFPWLLIIIFGIFGLTVMLFVFLFSKQQRIKMLILPPVPVPKIKGIDPDLLKEGKLEEVNTILAIHDSYKPEFHSDDSWVEFIELDIDEPDEKTEESDTDRLLSSDHEKSHSNLGVKDGDSGRTSCCEPDILETDFNANDIHEGTSEVAQPQRLKGEADLLCLDQKNQNNSPYHDACPATQQPSVIQAEKNKPQPLPTEGAESTHQAAHIQLSNPSSLSNIDFYAQVSDITPAGSVVLSPGQKNKAGMSQCDMHPEMVSLCQENFLMDNAYFCEADAKKCIPVAPHIKVESHIQPSLNQEDIYITTESLTTAAGRPGTGEHVPGSEMPVPDYTSIHIVQSPQGLILNATALPLPDKEFLSSCGYVSTDQLNKIMP

(19) United States
(12) Patent Application Publication
(10) Pub. No.: US 2011/0263501 A1
(43) Pub. Date: Oct. 27, 2011

(54) Growth Hormone Conjugate with Increased Stability

(75) Inventor: Nils Langeland Johansen, Bagsvaerd (DK)

(73) Assignee: Nono Nordisk Health Care AG, Zurich (CH)

(21) Appl. No.: 13/058,985

(22) PCT Filed: Sep. 9, 2009
(86) PCT No.: PCT/EP2009/061688

§ 371 Date: Jul. 8, 2011

(30) Foreign Application Priority Data
Sep. 9, 2008 (EP) 08163958.5

(51) Int. Cl. A61K 38/27; A61P 5/06; A61P 13/12; A61P 29/00; A61P 31/18; A61P 19/00; A61P 19/10; A61P 1/16; A61P 3/04; A61P 15/08; A61P 25/30; A61P 25/32; A61P 25/00; A61P 19/02; A61P 15/10; A61P 25/28; A61P 25/24; A61P 3/00; A61P 31/00; C12P 21/00; C07K 14/61

(52) U.S. Cl. 514/11.3; 530/399; 435/68.1

(57) ABSTRACT

Novel growth hormone conjugates comprising a growth hormone compound (GH) and a growth hormone binding protein (GHBP) are disclosed. The invention also encompasses a novel derivatization method for producing stable, long-lasting conjugate (hGH-GHBP) by site specific conjugation. The novel GH-GHBP conjugates have an extended half-life in circulation that facilitates therapeutic use of the protein. The GH-GHBP conjugates exhibit pharmacological properties such as increased functional in vivo half-life, improved renal filtration, improved protease protection and albumin binding.

### FIELD OF THE INVENTION

The present invention is related to a growth hormone conjugates and methods for preparing them. The conjugates described herein are growth hormone compounds conjugated to growth hormone binding protein by site-specific conjugation. The novel conjugates have an extended half-life in circulation, which facilitates therapeutic use of the protein.

### BACKGROUND OF THE INVENTION

Human growth hormone (hGH) is a protein of 191 amino acids length with disulphide bridges and a molecular weight of 22 kDa. The disulphide bonds link positions 53 and 165 and positions 182 and 189. hGH plays a key role in promoting growth, maintaining normal body composition, anabolism and lipid metabolism. It also has direct effects on intermediate metabolism, such as decreased glucose uptake, increased lipolysis, increased amino acid uptake and protein synthesis. The hormone also exerts effects on other tissues including adipose tissue, liver, intestine, kidney, skeleton, connective tissue and muscle. Recombinant hGH has been produced and commercially available as, for ex: Genotropin™ (Pharmacia Upjohn), Nutropin™ and Protropin™ (Genentech), Humatrope™ (Eli Lilly), Serostim™ (Serono) and Norditropin™ (Novo Nordisk). Additionally, an analogue with an additional methionine residue at the N-terminal end is also marketed as, for ex: Somatonorm™ (Pharmacia Upjohn/Pfizer).

Manipulation of a given protein is attempted especially if the said protein is of therapeutic nature so as to increase its stability or half-life. In this context, modification of the proteins by conjugating groups to the said protein is one of the ways that alter the properties of the protein. hGH has a short-half life thus necessitating at least three injections or in most cases daily injections in children suffering from growth deficiencies. The current hGH therapeutic regimen requires daily subcutaneous injections. This has created multiple hurdles to the patient which may include cost; ease in administering in addition to issues on the patient's tolerance for daily injections.

To enhance the stability, decrease the clearance rate and decrease the antigenicity of therapeutic proteins, various approaches have been proposed including chemical modification of the therapeutic protein. Different methods for creating long-lasting growth hormone include pegylation approach and development of sustained release formulations.

It has been described that the half-life of growth hormone (GH) in circulation can be significantly increased by covalent binding to growth hormone binding protein (GHBP) (G. Baumann et al., Metabolism-Clinical and Experimental 38(4), 330-333 (1989)). Unfortunately this conjugate is unsuited for clinical use because it's inherently heterogeneity and because it is very likely to be unable to stimulate the GH receptor. It has also been described that a GH-Linker-GHBP fusion protein has increased half-live in circulation (I. R. Wilkinson et al., Nat. Med. 13 (9), 1108-1113 (2007)). Unfortunately this approach also has its problems. It is so that in artificial fusion proteins potentially immunogenic “non self” sequences are created between linker and fusion partners. Such non-self sequences can cause immunogenecity which is highly undesirable for a drug for chronical use.

Use of transglutaminase (TGase) for pegylating growth hormone by post-translational conjugation of the hormone wherein transglutaminase is used to create a point of attachment at specific positions in the protein to which PEG is selectively attached has previously been described (WO06134148, WO05070468, US60/957732). In addition, EP950665 and EP785276 describe transglutaminase-mediated alteration of physiologically active proteins.

### SUMMARY OF THE INVENTION

Disclosed herein are novel modified forms of growth hormone having specific amino acid residues modified and bound to GHBP by site-specific conjugation. The disclosure also encompasses methods for preparing such compounds as well as pharmaceutical use of such compounds. The conjugates thus derived by the methods disclosed in the present invention have improved pharmacological properties compared to the corresponding unconjugated peptide. Examples of such pharmacological properties include increased functional in vivo half-life, decreased immunogenicity, improved renal filtration and protease protection, and albumin binding.

In one aspect the invention provides a composition of formula:

A-B-C

wherein
A is a radical of a growth hormone compound;
B is a linking and spacing bivalent residue;
C is a radical of an extracellular part of the growth hormone receptor monomer compound; and
- is a covalent bond,
   wherein the linkages between A and B and/or the linkage between B and C are not provided by recombinant expression of a nucleic acid comprising a nucleic acid sequence encoding for a fusion protein having the structure A-B-C.

The invention also provides a method of obtaining A-B-C comprising the steps of
(a) enzymatic derivatization of a growth hormone compound and
(b) conjugating the enzyme modified growth hormone compound to a growth hormone receptor monomer compound.

The invention also provides a method of treating disease states in a mammal that will benefit from increase in the activity of growth hormone by administering an effective amount of hGH-GHBP conjugate derived as described in the present invention.

### DESCRIPTION OF THE INVENTION

The present invention is related to protein conjugates between a protein, especially human growth hormone (hGH), and growth hormone binding protein (GHBP). One method described here for preparing such conjugates encompasses conjugation of hGH with GHBP by site-specific conjugation. The hGH-GHBP conjugates provided by this invention have enhanced pharmacological properties like stability, extended half-life in circulation, renal clearance as compared to hGH itself and reduced immunogenecity as compared to fusion proteins of hGH and hGH-GHBP as described in for instance Baumann et al., Metabolism-Clinical and Experimental 38(4), 330-333 (1989)) and I. R. Wilkinson et al., Nat. Med. 13 (9), 1108-1113 (2007). “Conjugate” as used herein is meant to indicate a fused or a modified protein or peptide, for example, a protein bound to another chemical moiety or another protein. Conjugation or conjugated means an activity or a process to form conjugates.

In one embodiment the invention provides a compound of formula:

A-B-C

wherein
A is a radical of a growth hormone compound;
B is a linking and spacing bivalent residue;
C is a radical of a growth hormone binding protein compound; and
- is a covalent bond,
   wherein the linkages between A and B and/or the linkage between B and C are not provided by recombinant expression of a nucleic acid comprising a nucleic acid sequence encoding a fusion protein having the structure A-B-C.

In one embodiment, the invention provides a compound of formula:

A-B-C
A is a radical of a growth hormone compound;
B is a linking and spacing bivalent residue;
C is a radical of a growth hormone binding protein compound; and
- is a covalent bond,
   wherein B is not a pure peptide chain.

In the present context, the term “compound” also encompasses pharmaceutically acceptable salts, prodrugs and solvates of said compound. In the present context, the term “pharmaceutically acceptable salt” is intended to indicate salts which are not harmful to the patient. Such salts include pharmaceutically acceptable acid addition salts, pharmaceutically acceptable metal salts, pharmaceutically acceptable ammonium salts and pharmaceutically acceptable alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. 1977, 66, 2, which is incorporated herein by reference. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like.

A growth hormone (GH) compound is a peptide comprising an amino acid sequence, which has at least 80% identity to SEQ ID No. 1 and which peptide has an activity in the assay described in Example 7 of at least 10% of hGH (in other words that the EC₅₀ of the GH compound is less than 100× EC₅₀ of hGH). In one embodiment, the activity of the GH compound is at least 20%, such as at least 30%, for instance at least 40%, such as at least 50%, for instance at least 60%, such as at least 70%, for instance at least 80%, such as at least 90% of hGH, for instance substantially the same activity as hGH.

In one embodiment, the growth hormone compound is a peptide comprising an amino acid sequence having at least 85%, such as at least 90%, for instance at least 95%, such as at least 96%, for instance at least 97%, such as at least 98%, for instance at least 99% identity to SEQ ID No. 1. In one embodiment, the growth hormone compound is a fragment of such a peptide, which fragment has retained a significant amount of the growth hormone activity as described above.

In one embodiment, the growth hormone compound is a peptide comprising an amino acid sequence, which sequence is at least 90%, for instance at least 95%, such as at least 96%, for instance at least 97%, such as at least 98%, for instance at least 99% similar to SEQ ID No. 1.

In one embodiment, the growth hormone compound is a growth hormone analogue as described in WO2006048777, WO2004022593, WO2005018659, WO2005074524; WO2005074546, WO2005074650, U.S. Pat. No. 5,849,535, U.S. Pat. No. 6,136,563, U.S. Pat. No. 6,022,711, or U.S. Pat. No. 6,143,523.

In one embodiment, the growth hormone compound is hGH.

The term “peptide” is intended to indicate a sequence of two or more amino acids joined by peptide bonds, wherein said amino acids may be natural or unnatural. The term encompasses the terms polypeptides and proteins, which may consists of two or more polypeptides held together by covalent interactions, such as for instance cysteine bridges, or non-covalent interactions. It is to be understood that the term is also intended to include peptides, which have been derivatized, for instance by the attachment of lipophilic groups, PEG or prosthetic groups. The term peptide includes any suitable peptide and may be used synonymously with the terms polypeptide and protein, unless otherwise stated or contradicted by context; provided that the reader recognize that each type of respective amino acid polymer-containing molecule may be associated with significant differences and thereby form individual embodiments of the present invention (for example, a peptide such as an antibody, which is composed of multiple polypeptide chains, is significantly different from, for example, a single chain antibody, a peptide immunoadhesin, or single chain immunogenic peptide). Therefore, the term peptide herein should generally be understood as referring to any suitable peptide of any suitable size and composition (with respect to the number of amino acids and number of associated chains in a protein molecule). Moreover, peptides described herein may comprise non-naturally occurring and/or non-L amino acid residues, unless otherwise stated or contradicted by context.

The term peptide, unless otherwise stated or contradicted by context, (and if discussed as individual embodiments of the term(s) polypeptide and/or protein) also encompasses derivatized peptide molecules. Briefly, in the context of the present invention, a derivative is a peptide in which one or more of the amino acid residues of the peptide have been chemically modified (for instance by alkylation, acylation, ester formation, or amide formation) or associated with one or more non-amino acid organic and/or inorganic atomic or molecular substituents (for instance a polyethylene glycol (PEG) group, a lipophilic substituent (which optionally may be linked to the amino acid sequence of the peptide by a spacer residue or group such as β-alanine, γ-aminobutyric acid (GABA), L/D-glutamic acid, succinic acid, and the like), a fluorophore, biotin, a radionuclide, etc.) and may also or alternatively comprise non-essential, non-naturally occurring, and/or non-L amino acid residues, unless otherwise stated or contradicted by context (however, it should again be recognized that such derivatives may, in and of themselves, be considered independent features of the present invention and inclusion of such molecules within the meaning of peptide is done for the sake of convenience in describing the present invention rather than to imply any sort of equivalence between naked peptides and such derivatives). Non-limiting examples of such amino acid residues include for instance 2-aminoadipic acid, 3-amino-adipic acid, β-alanine, β-aminopropionic acid, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, 2,4-diaminobutyric acid, desmosine, 2,2′-diaminopimelic acid, 2,3-diaminopropionic acid, N-ethylglycine, N-ethylasparagine, hydroxylysine, allohydroxylysine, 3-hydroxyproline, 4-hydroxyproline, isodesmosine, alloisoleucine, N-methylglycine, N-methylisoleucine, 6-N-methyllysine, N-methylvaline, norvaline, norleucine, ornithine, and statine halogenated amino acids. It is to be understood that this derivatization is not a derivatization of the present invention, but rather a derivatization already present on the growth hormone compound before the conjugation of the present invention, or a derivatization performed after the conjugation of the present invention.

The term “identity” as known in the art, refers to a relationship between the sequences of two or more peptides, as determined by comparing the sequences. In the art, “identity” also means the degree of sequence relatedness between peptides, as determined by the number of matches between strings of two or more amino acid residues. “Identity” measures the percent of identical matches between two or more sequences and the other, with gap alignments (if any) addressed by a particular mathematical model or computer program (i.e., “algorithms”). Identity of related peptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

Preferred methods to determine identity are designed to give the largest match between the sequences tested. Methods to determine identity are described in publicly available computer programs. Preferred computer program methods to determine identity between two sequences include the GCG program package, including GAP (Devereux et al., Nucl. Acid. Res. 12, 387 (1984); Genetics Computer Group, University of Wisconsin, Madison, Wis.), BLASTP, BLASTN, Muscle (Edgar, Robert C., Nucleic Acids Research 32(5), 1792-97 ((2004)), clustal X and clustal W (Larkin M A et al., Bioinformatics 23, 2947-2948 (2007) and tcoffee (Notredame, C., Journal of Molecular Biology 302, 205-217 (2000). The BLASTX program is publicly available from the National Center for Biotechnology Information (NCBI) and other sources (BLAST Manual, Altschul et al. NCB/NLM/NIH Bethesda, Md. 20894; Altschul et al., supra). The well known Smith Waterman algorithm may also be used to determine identity.

For example, using the computer algorithm GAP (Genetics Computer Group, University of Wisconsin, Madison, Wis.), two peptides for which the percent sequence identity is to be determined are aligned for optimal matching of their respective amino acids (the “matched span”, as determined by the algorithm). A gap opening penalty (which is calculated as 3.times. the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. A standard comparison matrix (see Dayhoff et al., Atlas of Protein Sequence and Structure, vol. 5, supp. 3 (1978) for the PAM 250 comparison matrix; Henikoff et al., Proc. Natl. Acad. Sci USA 89, 10915-10919 (1992) for the BLOSUM 62 comparison matrix) is also used by the algorithm.

Preferred parameters for a peptide sequence comparison include the following:

Algorithm: Needleman et al., J. Mol. Biol. 48, 443-453 (1970); Comparison matrix: BLOSUM 62 from Henikoff et al., PNAS USA 89, 10915-10919 (1992); Gap Penalty: 12, Gap Length Penalty: 4, Threshold of Similarity: 0.

The GAP program is useful with the above parameters. The aforementioned parameters are the default parameters for peptide comparisons (along with no penalty for end gaps) using the GAP algorithm.

The term “similarity” is a concept related to identity, but in contrast to “identity”, refers to a sequence relationship that includes both identical matches and conservative substitution matches. If two polypeptide sequences have, for example, (fraction ( 10/20)) identical amino acids, and the remainder are all non-conservative substitutions, then the percent identity and similarity would both be 50%. If, in the same example, there are 5 more positions where there are conservative substitutions, then the percent identity remains 50%, but the percent similarity would be 75% ((fraction ( 15/20))). Therefore, in cases where there are conservative substitutions, the degree of similarity between two polypeptides will be higher than the percent identity between those two polypeptides.

Conservative modifications a peptide comprising an amino acid sequence of SEQ ID No. 1 (and the corresponding modifications to the encoding nucleic acids) will produce peptides having functional and chemical characteristics similar to those of a peptide comprising an amino acid sequence of SEQ ID No. 1. In contrast, substantial modifications in the functional and/or chemical characteristics of peptides according to the invention as compared to a peptide comprising an amino acid sequence of SEQ ID No. 1 may be accomplished by selecting substitutions in the amino acid sequence that differ significantly in their effect on maintaining (a) the structure of the molecular backbone in the area of the substitution, for example, as a sheet or helical conformation, (b) the charge or hydrophobicity of the molecule at the target site, or (c) the bulk of the side chain.

For example, a “conservative amino acid substitution” may involve a substitution of a native amino acid residue with a nonnative residue such that there is little or no effect on the polarity or charge of the amino acid residue at that position. Furthermore, any native residue in the polypeptide may also be substituted with alanine, as has been previously described for “alanine scanning mutagenesis” (see, for example, MacLennan et al., Acta Physiol. Scand. Suppl. 643, 55-67 (1998); Sasaki et al., Adv. Biophys. 35, 1-24 (1998), which discuss alanine scanning mutagenesis).

Desired amino acid substitutions (whether conservative or non-conservative) may be determined by those skilled in the art at the time such substitutions are desired. For example, amino acid substitutions can be used to identify important residues of the peptides according to the invention, or to increase or decrease the affinity of the peptides described herein for the receptor in addition to the already described mutations.

Naturally occurring residues may be divided into classes based on common side chain properties:

1) hydrophobic: norleucine, Met, Ala, Val, Leu, Iie;

2) neutral hydrophilic: Cys, Ser, Thr, Asn, Gln;

3) acidic: Asp, Glu;

4) basic: His, Lys, Arg;

5) residues that influence chain orientation: Gly, Pro; and

6) aromatic: Trp, Tyr, Phe.

In making such changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al., J. Mol. Biol., 157, 105-131 (1982). It is known that certain amino acids may be substituted for other amino acids having a similar hydropathic index or score and still retain a similar biological activity. In making changes based upon the hydropathic index, the substitution of amino acids whose hydropathic indices are within .±2 is preferred, those that are within ±1 are particularly preferred, and those within ±0.5 are even more particularly preferred. The greatest local average hydrophilicity of a protein, as governed by the hydrophilicity of its adjacent amino acids, correlates with its immunogenicity and antigenicity, i.e., with a biological property of the protein.

The following hydrophilicity values have been assigned to amino acid residues: arginine (+3.0); lysine ('3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids whose hydrophilicity values are within ±2 is preferred, those that are within ±1 are particularly preferred, and those within ±0.5 are even more particularly preferred.

Peptides of the present invention may also include non-naturally occurring amino acids.

In one embodiment, B does not comprise a peptide chain. A peptide chain in the context of the present invention is to be understood as continous peptide bonds, that is a chain made up of amino acid residues linked together by amide bonds.

It is to be understood that the actual structure of B is not an essential feature of the invention. The length and nature of the linker is to be determined by the person skilled in the art when optimizing the biological profile of the conjugate of the present invention.

B may for instance comprise at least 10 covalent bonds, B may also be made up of repeating C—S—C, C—O—C, or C—C—C units, such as for instance in form of a polyethylene glycol molecule (PEG). The term “polyethylene glycol”, “Peg” or “PEG” (poly(ethylene glycol)) means a polydisperse or monodisperse diradical of the structure

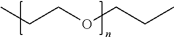

wherein n is an integer larger than 1, and its molecular weight can range from between approximately 100 Da to approximately 1,000,000 kDa or even larger. For the purpose of the present invention, B could be a PEG of a size of for instance from around 500, to around 50,000 kDa, such as for instance around 500, 750, 1,000, 2,000, 5,000, 10,000, 20,000, 30,000, 40,000 or 50,000 kDa, such as between 1,000 and 10,000.

More often than not, the structure of B is a consequence of the choice of method for conjugating C to A as illustrated in the examples.

C is a radical of a growth hormone receptor monomer compound, or rather of a GHBP compound. GHBP is the soluble extracellular portion of the GH receptor, most likely derived by proteolytic cleavage of the growth hormone receptor. This protein binds 40-50% of circulating GH and seems to protect GH from elimination and degradation, thus regulating GH action. Under normal physiological conditions, GHBP is complexed with about half of the GH in human plasma (See Baumann et al., Endocrinol 122, 976-984 (1988)) and acts as a reservoir or a buffer, damping the oscillations of plasma GH, prolonging GH half-life, and modulating GH bioactivity through competition with GHR for GH (Baumann et al., J Endocrinol Invest. 17, 67 (1994)). GHBP is 638 amino acids long and is provided as SEQ ID No. 2.

A GHBP compound is a peptide comprising an amino acid sequence, which has at least 80% identity to SEQ ID No. 2 and which peptide is capable of binding to growth hormone with a KD<100 nM. In one embodiment, the GHBP compound is a peptide comprising an amino acid sequence having at least 85%, such as at least 90%, for instance at least 95%, such as at least 96%, for instance at least 97%, such as at least 98%, for instance at least 99% identity to SEQ ID No. 2. In one embodiment, the GHBP compound comprises the sequence of GHBP (SEQ ID No. 2). In one embodiment, the GHBP compound is a fragment of such a peptide, which fragment has retained a significant amount of the GHBP activity (that is, capable of binding to GH), such as having substantially the same GHBP activity, of such a peptide. In one embodiment, the GHBP compound has a Serine in the N-terminal.

In the present invention, the linkages between A and B and/or the linkage between B and C are not provided by recombinant expression of a nucleic acid comprising a nucleic acid sequence encoding for a fusion protein having the structure A-B-C. In other words, the linkages between A and B and B and C are established by modifications performed after the expresssion (or generation) of the compounds of the growth hormone compound and the GHBP compound. Such posttranslational modification is well known in the art and includes for instance acylation, alkylation (ex: methyl, ethyl), amidation, biotinylation, formylation, glycosylation and phosphorylation.

In one embodiment, A-B-C is not a linear peptide. In one embodiment, B is attached to the N-terminal of the GH compound. In one embodiment, B is attached to the C-terminal of the GH compound. In one embodiment, B is attached to an amino acid side-chain of the GH compound. In one embodiment, B is attached to the N-terminal of the GHBP compound. In one embodiment, B is attached to the C-terminal of the GHBP compound. In one embodiment, B is attached to an amino acid side chain of the GHBP compound.

In one embodiment, at least one of the covalent bonds establised in the preparation of a GH-GHBP conjugate of the present invention is prepared by use of an enzyme as illustrated in the examples. Such an enzyme may for instance be selected from the group consisting of transglutaminases, serine proteases and cysteine proteases. In one embodiment, said enzyme is a transglutaminase. Such transglutaminase may for instance be selected from the group consisting of microbial transglutaminases, tissue transglutaminases and factor XIII and variants thereof. In one embodiment, said enzyme is a cysteine protease. Such a cysteine protease may for instance be selected from the group consisting of papain, sortase A and sortase B. In one embodiment, said enzyme is a serine protease. Such a serine protease may for instance be selected from the group consisting of carboxypeptidase Y (CPY) (PCT application WO2005/035553 contains general disclosure of protein modification using CPY), trypsin and chymotrypsin.

The compounds of the present invention may be prepared by many different methods, an exemplary selection of which, which is not to be considered as limiting, are shown below.

The present invention also provides methods for preparing GH-GHBP conjugates of the present invention.

As stated above, at least one of the covalent bonds establised in the preparation of a GH-GHBP conjugate of the present invention may be prepared by use of a transglutaminase. Transglutaminases may include microbial transglutaminases such as that isolated from the Streptomyces species; S. mobaraense, S. cinnamoneum, S. griseocarneum (U.S. Pat. No. 5,156,956 incorporated herein by reference), S. lavendulae (U.S. Pat. No. 5,252,469 incorporated herein by reference) and Streptomyces ladakanum (JP2003199569 incorporated herein by reference). Other useful microbial transglutaminases have been isolated from Bacillus subtilis (disclosed in U.S. Pat. No. 5,731,183, which is incorporated herein by reference) and from various Myxomycetes. Other examples of useful microbial transglutaminases are those disclosed in WO 96/06931 (e.g. transglutaminase from Bacillus lydicus) and WO 96/22366, both of which are incorporated herein by reference. Useful non-microbial transglutaminases include guinea-pig liver transglutaminase, and transglutaminases from various marine sources like the flat fish Pagrus major (disclosed in EP-0555649, which is incorporated herein by reference), and the Japanese oyster Crassostrea gigas (disclosed in U.S. Pat. No. 5,736,356, which is incorporated herein by reference). Functional analogues and derivatives thereof may also be useful.

In one embodiment, the TGase used in the methods of the invention is a microbial transglutaminase. In one embodiment, the TGase is from S. mobaraense or a variant thereof, for instance as described in WO2007/020290 and WO2008/020075. In one embodiment, the TGase is from S. ladakanum or a variant thereof, for instance as described in WO2008/020075.

The conjugation of hGH to GHBP according to the present invention may be achieved by TGase-mediated modification leading to alteration at specific lysine (Lys) or glutamine (Glu) positions of in the sequence of the GH compound. hGH (SEQ ID No. 1) has 9 lysine residues at positions 38, 41, 70, 115, 140, 145, 158, 168 and 172 and 13 glutamine residues at positions 22, 29, 40, 46, 49, 68, 69, 84, 91, 122, 137, 141 and 181. Not all of these are readily available for modification. In one embodiment, GHBP in the present invention is extended in the N-terminus with a serine to form compound of formula IV. The process of N-terminal extension of a protein with serine can be readily recognized by a person skilled in the art (See Sundstrom et al, J Biol Chem 271, 32197 (1996); Fuh et al, J Biol Chem. 265, 3111 (1990).

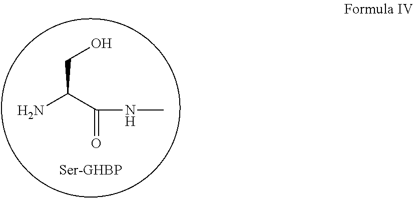

TGase-mediated enzymatic modification can be exemplified as follows:

R′—CONH₂+H₂N—R″→R′—CONH—R″+NH₃,

wherein R′—CONH₂ and H₂N—R″ are substrates for the enzymatic reactions. R′ and R″ do not have to comprise protein parts, but R′ and R″ does need to have some kind of structure which enables the enzyme to recognize R′—CONH₂ and H₂N—R″ as substrates.

In one embodiment, R′—CONH₂ is a peptide, such as a growth hormone compound, and H₂N—R″ is a nucleophile, for instance as described in WO2005/070468, and WO2006/134148, which are herein incorporated by reference in their entirety. In one embodiment, H₂N—R″ comprises B as described above, for instance in the form of H-B-H, so that B is a divalent radical of H₂N—R″.

In one embodiment, R′—CONH₂ is a peptide, such as a growth hormone compound, and H₂N—R″ is a nucleophile, for instance as described in WO2008/003750, which is herein incorporated by reference in its entirety. In one embodiment, H₂N—R″ comprises B as described above, or a group, which can be modified so that the resulting compound comprises B as described above,

In one embodiment, H₂N—R″ is a peptide, such as a growth hormone compound, which is reacted with R′—CONH₂, for instance as described in US60/957732, which is herein incorporated by reference in its entirety.

Several examples of different, functional R′—CONH₂ and H₂N—R″ compounds are shown below, and it is clear that the structure of B should not be limiting for the present invention. More examples of possibles structures of B can be found in for instance WO2005/070468, WO2006/134148, WO2008/003750 and US60/957732.

In WO2005/070468 (and WO2006/134148), it is stated that a compound corresponding to H₂N—R″ (when R′ is a peptide) could be of the following formula

H₂N-D-R″—X

wherein
D represents a bond or oxygen;
R′″ represents a linker (termed the R′″ linker) or a bond;
X represents a radical comprising a functional group or a latent functional group not accessible in the amino acid residues constituting the peptide R¹—C(═O)—NH₂.

In one embodiment, D represents —O—.

In one embodiment, D represents a single bond.

In one embodiment, the functional group or latent functional group comprised in X is selected from, or can be activated to, a functional group selected from the group of the keto-, aldehyde-, —NH—NH₂, —O—C(═O)—NH—NH₂, —NH—C(═O)—NH—NH₂, —NH—C(═S)—NH—NH₂, —NHC(═O)—NH—NH—C(═O)—NH—NH₂, —NH—NH—C(═O)—NH—NH₂, —NH—NH—C(═S)—NH—NH₂, —NH—C(═O)—C₆H₄—NH—NH₂, —C(═O)—NH—NH₂, —O—NH2, —C(═O)—O—NH₂, —NH—C(═O)—O—NH₂, —NH—C(═S)—O—NH₂, alkynyl, azide and nitril-oxide. In one embodiment, the functional group or latent functional group comprised in X is selected from, or can be activated to, one of the groups below:

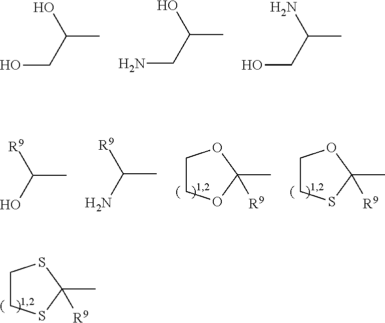

wherein R⁹ is selected amongst H, C₁₋₆alkyl, aryl and heteroaryl.

The term “alkyl” is intended to indicate a monovalent radical of an alkane. The term “alkylene” is intended to indicate a divalent radical of an alkane. The term “alkane” is intended to indicate a saturated, linear, branched and/or cyclic hydrocarbon. Unless specified with another number of carbon atoms, the term is intended to indicate hydrocarbons with from 1 to 30 (both included) carbon atoms, such as 1 to 20 (both included), such as from 1 to 10 (both included), e.g. from 1 to 6 (both included); or from 15 to 30 carbon atoms (both included).

The term “alkenyl” is intended to indicate a monovalent radical of an alkene. The term “alkenylene” is intended to indicate a monovalent radical of an alkene. The term “alkene” is intended to a indicate linear, branched and/or cyclic hydrocarbon comprising at least one carbon-carbon double bond. Unless specified with another number of carbon atoms, the term is intended to indicate hydrocarbons with from 2 to 30 (both included) carbon atoms, such as 2 to 20 (both included), such as from 2 to 10 (both included), e.g. from 2 to 5 (both included); or from 15 to 30 carbon atoms (both included).

The term “alkynyl” is intended to indicate a monovalent radical of an alkyne. The term “alkynylene” is intended to indicate a divalent radical of an alkyne. The term “alkyne” is intended to indicate a linear, branched and/or cyclic hydrocarbon comprising at least one carbon-carbon triple bond, and it may optionally comprise one or more carbon-carbon double bonds. Unless specified with another number of carbon atoms, the term is intended to indicate hydrocarbons with from 2 to 30 (both included) carbon atoms, such as from 2 to 20 (both included), such as from 2 to 10 (both included), e.g. from 2 to 6 (both included); or from 15 to 30 carbon atoms (both included).

The terms “heteroalkane”, “heteroalkene” and “heteroalkyne” is intended to indicate alkanes, alkenes and alkynes as defined above, in which one or more hetero atom or group have been inserted into the structure of said moieties. Examples of hetero groups and atoms include —O—, —S—, —S(═O)—, —S(═O)2-, —C(═O)— —C(═S)— and —N(R*)—, wherein R* represents hydrogen or C₁₋₆-alkyl. Consequently. heteroalkylene, heteroalkenylene and heteroalkynylene are divalent radicals of heteroalkane, heteroalkene and heteroalkyne respectively. Examples of heteroalkanes include:

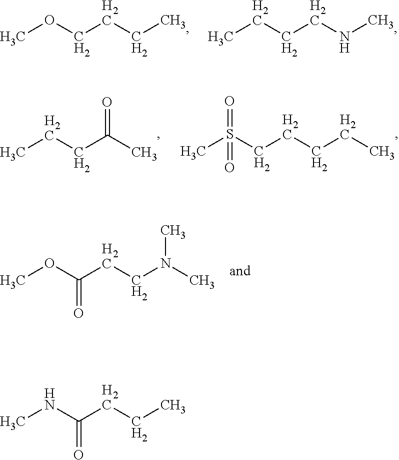

The term “arylene” is intended to indicate a bivalent radical of an aryl. The term “aryl” is intended to indicate a homocyclic aromatic ring radical or a fused homocyclic ring system radical wherein at least one of the rings are aromatic. Typical aryl groups include phenyl, biphenylyl, naphthyl, tetralinyl and the like.

The term “heteroarylene” is intended to indicate a bivalent radical of a heteroaryl. The term “heteroaryl” is intended to indicate an aromatic ring radical with for instance 5 to 7 ring atoms, or to a fused aromatic ring system radical with for instance from 7 to 18 ring atoms, wherein at least on ring is aromatic and contains one or more heteroatoms as ring atoms selected from nitrogen, oxygen, or sulfur heteroatoms, wherein N-oxides and sulfur monoxides and sulfur dioxides are permissible heteroaromatic substitutions. Examples include furanyl, thienyl, thiophenyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, quinolinyl, isoquinolinyl, benzofuranyl, benzothiophenyl, indolyl, and indazolyl, and the like.

The R′″ linker indicates a moiety functioning as a means to separate X from NH₂-D- and can in many ways be viewed as a linker equivalent to B. One function of the linker R′″ is to provide adequate flexibility in the linkage between the peptide and the GHBP to be attached to X in a later step. Typical examples of R′″ include straight, branched and/or cyclic C₁₋₁₀-alkylene, C₂₋₁₀-alkenylene, C₂₋₁₀-alkynylene, C₂₋₁₀-heteroalkylene, C₂₋₁₀-heteroalkenylene, C₂₋₁₀-heteroalkynylene, wherein one or more homocyclic aromatic compound biradicals or heterocyclic compound biradicals may be inserted. Particular examples of R′″ include

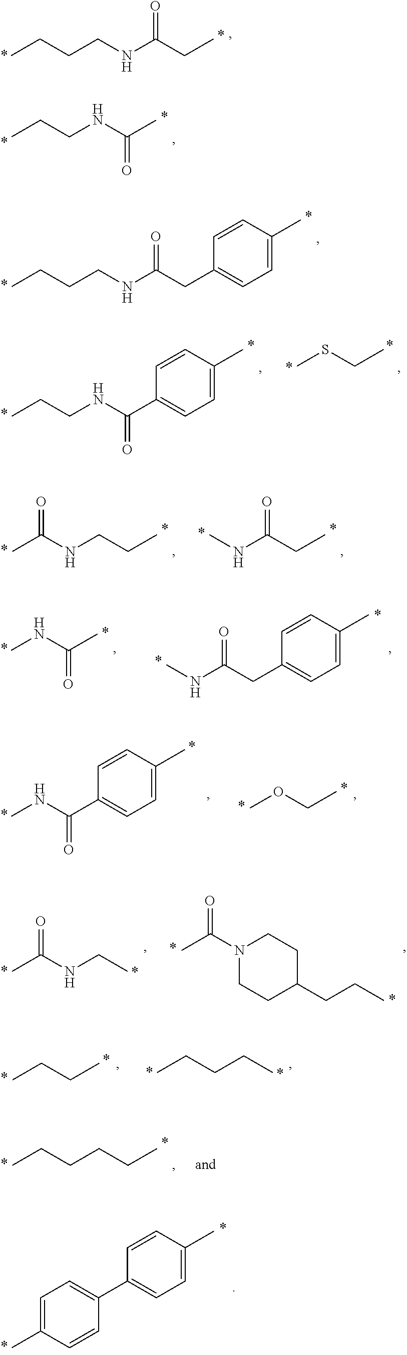

wherein * denotes points of attachment.

In one embodiment, R′″ represents —(CH₂)₄—CH(NH₂)—CO—NH—CH₂— or —(CH₂)₄—CH(NHCOCH₃)—CO—NH—CH₂—. In one embodiment, R′″ represents C₁₋₆-alkylene. In one embodiment, R′″ represents C₁₋₃-alkylene. In one embodiment, R′″ represents methylene or propylene.

In a subsequent step, the modifying group is then attached by reaction between the X group and a compound comprising the modifying group, wherein said compound also comprises a group, which can react selectively with the X group. According to the present invention, such modifying group is a radical of a growth hormone binding protein compound.

For more details, please refer to WO2005/070468 and/or WO2006/134148.

In WO2008/003750, it is stated that the compound corresponding to H₂N—R″ (when R′ is a peptide) is an aniline or heteroarylamine is of the formula

R^{c}—R^{b}—R^{a}—NH₂

wherein
R^{a} represents arylene or a heteroarylene, optionally substituted with a C₁₋₆-alkyl, halogen, cyano, nitro, hydroxyl, carboxyl, or aryl group;
R^{b} represents a bond or a linker, wherein said linker comprises a diradical selected from the group consisting of —C(═O)—NH—, —NH—, —O—, —S—, —O—P(O)(OH)—O—, —O—C(═O)—NH—, —NH—C(═O)—NH—, —(CH₂)₁₋₁₀—, —O—(CH₂)₃—NH—C(═O)—, —C(═O)NH(CH₂)₂₋₃₀—, —(CH₂)₁₋₃₀—C(═O)NH(CH₂)₂₋₃₀—, —(CH₂)₀₋₃₀—C(═O)NH—(CH2CH2O)1-10-(CH₂)₁₋₅—C(═O)—, —C(═O)NH—[(CH₂CH₂O)₁₋₁₀—(CH₂)₁₋₅—C(═O)]₁₋₅NH(CH₂)₂₋₃₀—, —C(═O)—, —(CH₂)₁₋₃₀—NHC(═O)—, —(CH₂)₁₋₃₀C(═O)—, —NHC(═O)NH(CH₂)₂₋₃₀—, —(CH₂)₁₋₃₀—NHC(═O)NH(CH₂)₂₋₃₀—, —(CH₂)₀₋₃₀C(═O)NH(CH₂)₂₋₃₀—NHC(═O)—(CH₂)₀₋₃₀—, —(CH₂)₀₋₃₀C(═O)NH(CH₂CH₂O)₁₋₃₀—CH₂CH₂NHC(═O)—(CH₂)₀₋₃₀—, or —NH(CH₂)₂₋₃₀—,

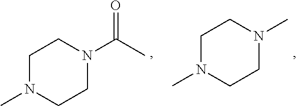

and combinations thereof, and
R^{c} represents a radical of a property-modifying group, wherein the term “property-modifying group” is intended to indicate a chemical group, which, when attached to the peptide in question alters one or more of the physicochemical or pharmacological properties of the peptide. Such properties could be solubility, tissue- and organ distribution, lipophilicity, susceptibility to degradation by various proteases, affinity to plasma proteins, such as albumin, functional in vivo half-life, plasma in vivo half-life, mean residence time, clearance, immunogenicity, and renal filtration. It is well-known in the art, that several types of chemical groups may have such property-modifying effects. According the present invention, the property-modifying group is a radical of GHBP as described above.

In one embodiment, R^{a} represents arylene or a heteroarylene, optionally substituted with a C₁₋₆-alkyl, halogen, cyano, nitro, hydroxyl, carboxyl, or a C₅₋₂₂-aryl group. In one embodiment, Ra represents arylene or a heteroarylene, optionally substituted with a C₁₋₆-alkyl, halogen, cyano, nitro, hydroxyl, carboxyl, or an C₆₋₁₈-aryl group. In one embodiment, Ra represents arylene or a heteroarylene, optionally substituted with a C₁₋₆-alkyl, halogen, cyano, nitro, hydroxyl, carboxyl, or C₄₋₁₆-aryl group. In one embodiment, Ra represents arylene or a heteroarylene, optionally substituted with a C₁₋₆-alkyl, halogen, cyano, nitro, hydroxyl, carboxyl, or C₆₋₈-aryl group. In one embodiment, R^{a} represents a C₅₋₂₂-arylene, optionally substituted as described above. In one embodiment, R^{a} represents a C₆₋₁₈-arylene, optionally substituted as described above. In one embodiment, R^{a} represents a C₆₋₁₄-arylene, optionally substituted as described above. In one embodiment, R^{a} represents a C₅₋₂₂-heteroarylene, optionally substituted as described above. In one embodiment, R^{a} represents a C₆₋₁₈-heteroarylene, optionally substituted as described above. In one embodiment, R^{a} represents a C₆₋₁₄-heteroarylene, optionally substituted as described above. In one embodiment, Ra represents a C₆₋₈-arylene, a C₁₂₋₁₈-arylene or a C₅₋₁₈-heteroarylene, optionally substituted as described above. In one embodiment, R^{a} represents a phenylene or a pyridylene group. In one embodiment, R^{a} represents 1,4-phenylene.

In one embodiment, R^{b} represents a bond, —C(═O)—NH—, or

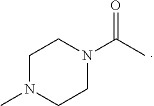

In one embodiment, R^{b} represents —C(═O)—NH—.

For more details, please refer to WO2008/003750,

In US60/957732 (and the PCT application embodimenting priority from US60/957732), it is stated that the compound conjugated to hGH and used for attachment of the property modifying group may be of the general formula: R⁷-Gln-Gly-R⁸, wherein R⁷ and R⁸ are desired substituents, where at least one of them comprises a chemical group that is suitable for further modification.

In one embodiment, the compound conjugated to hGH and used for attachment of the property modifying group has the formula:

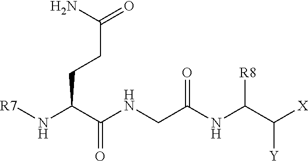

In one embodiment, R⁷ is

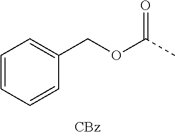

In one embodiment, R⁷ is CBz; R⁸ is selected from H, propargyl or 4-aminobenzyl; Y is OH or ═O; and X is CH₂OH or OH. In one embodiment, R⁷ is CBz, R⁸ is H, Y is OH and X is CH₂OH. In one embodiment, R⁷ is CBz, R⁸ is 4-aminobenzyl, Y is ═O, and X is OH. In one embodiment, R⁷ is CBz, R⁸ is propargyl, Y is ═O, and X is OH. Examples of such compounds are shown below

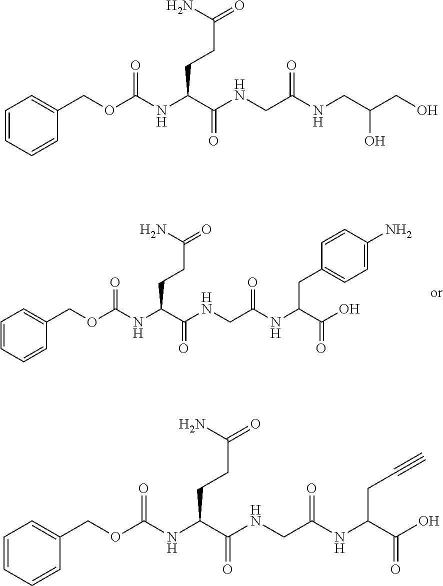

In one embodiment, cysteine protease is used for the enzyme-mediated modification of the proteins. Cysteine protease has a catalytic mechanism that involves a nucleophilic cysteine thiol in a catalytic triad. The cysteine protease of the invention can be selected from the group consisting of papain, sortase A and sortase B. The invention also provides serine protease for modifying the polypeptide. In one embodiment, the serine protease is CPY, trypsin or chymotrypsin.

As above, the compounds to be conjugated should be of a structure that makes them substrates for the particular enzymes. PCT application WO2005/035553 (and WO2006/084888) describes a general structure for substrates for CPY.

Glycan moieties on the compounds to be conjugated may also be used for conjugation. If the glycan is of the biantenna complex type, sialic acids may be oxidized selectively under mild conditions to generate reactive aldehydes as described in WO2008025856. These may then be used for chemical conjugation to a compound which is functionalized with moiety capable of reacting with an aldehyde. Reactive aldehydes may also be generated by enzymatic oxidation of galactose residues using galactose oxidase as described in WO2005014035. The complex glycan will optionally need trimming with sialidase, or galactosylation with galactosyltransferase and UDP-Gal, before reaction with galactose oxidase. In still another embodiment, UDP-Gal functionalized with an alkyne derivative is transferred to the biantenna glycan, and subsequently used for conjugation to a hGH molecule derivatized with an azido group as described in WO2006035057.

If there are no existing glycans on the compounds to be conjugated or they are not found suitable for conjugation, new N-glycosylation sites may be engineered into GHBP by incorporating the Asn-XXX-Thr/Ser consensus site into the peptide sequence by directed mutagenesis.

### Chemistry I

In one embodiment, the hGH-GHBP conjugate of the present invention may be prepared as illustrated below:

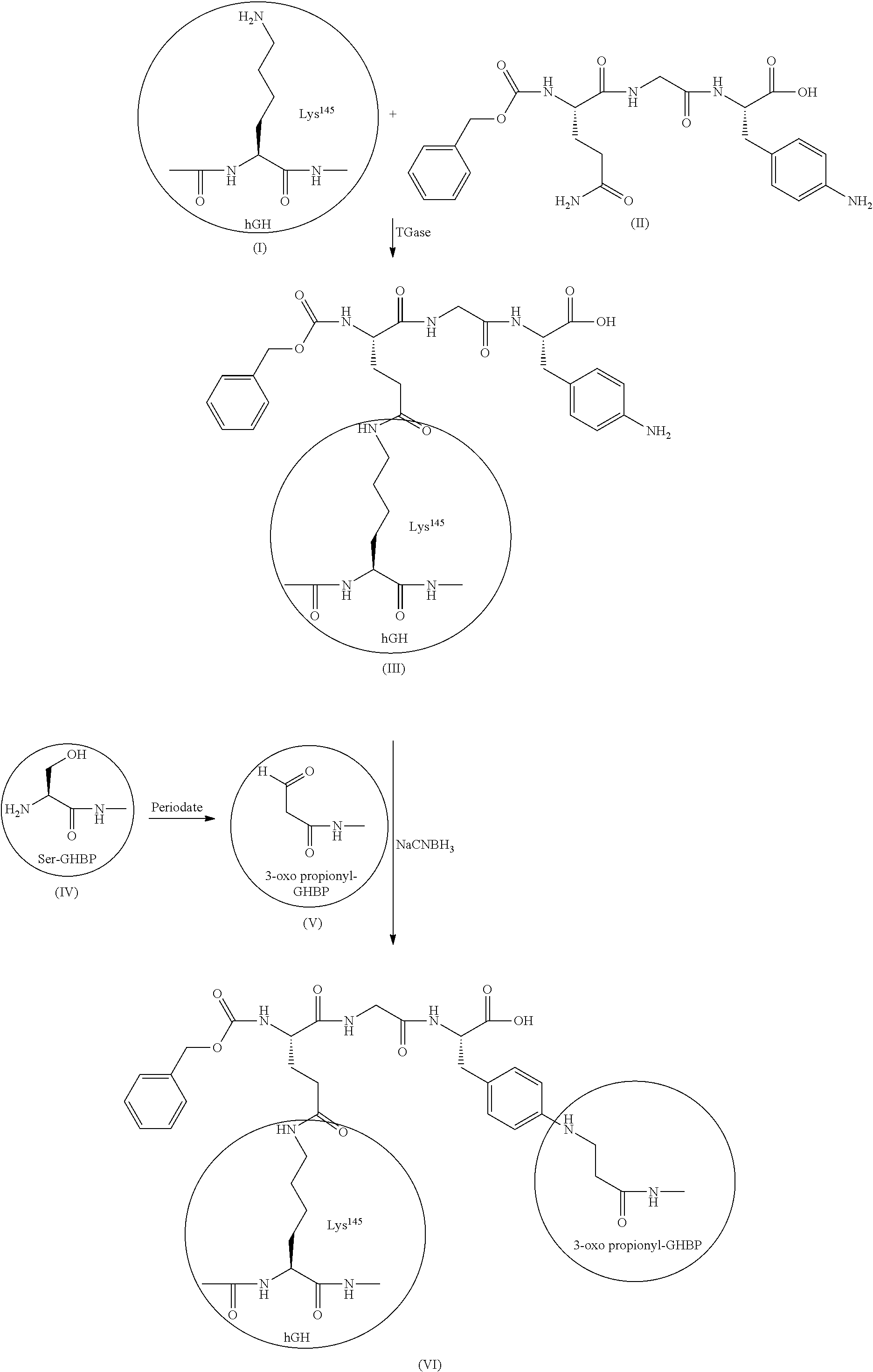

Peptide-bound lysine is acting as the amine donor affording cross-bonding of peptides.

In one embodiment, Lys145 of hGH (SEQ ID No. 1) is selectively modified. In one embodiment, at least two Lys-residues of hGH are modified.

### Chemistry II

In one embodiment, the invention teaches treatment of an aldehyde or ketone derived from the peptide compound with a property-modifying group-derived aniline or heteroarylamine to yield an imine or a hemiaminal. In one embodiment, aldehyde derived from the peptide compound is treated with property-modifying group-derived aniline or heteroarylamine.

The term “peptide-derived aldehyde (or ketone)” or “aldehyde (or ketone) derived from a peptide” is intented to indicate a peptide to which an aldehyde or ketone functional group has been covalently attached, or a peptide on which an aldehyde or ketone functional group has been generated. The preparation of peptide-derived aldehydes is well known to those skilled in the art, and any of these known procedures may be used to prepare the peptide-derived aldehyde required for the realization of the invention disclosed herein.

In one embodiment, the conjugate hGH-GHBP is prepared as illustrated below:

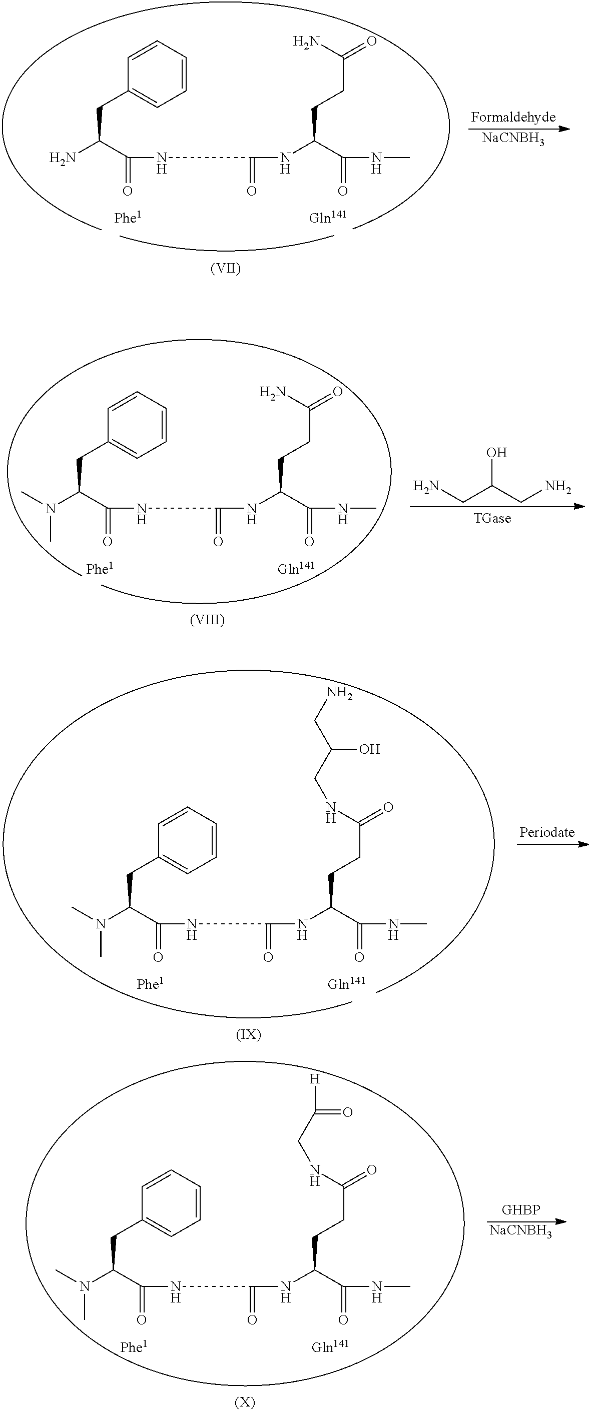

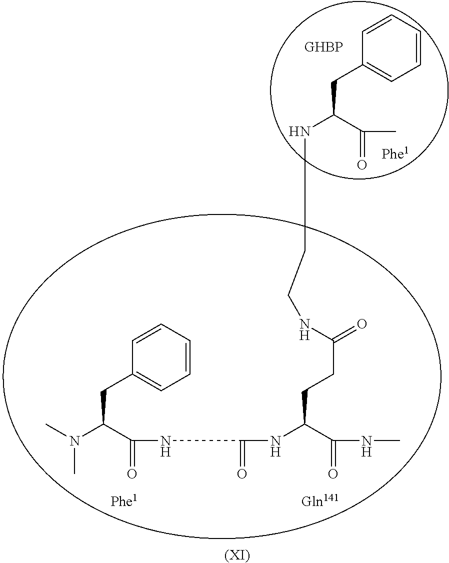

The process utilizes blocking of hGH's potential for reaction with aldehydes. If small unhindered aldehydes (as formaldehyde) are used then di-alkylation is likely to happen. Alternatively a hindered aldehyde can be used. In both cases further alkylation cannot take place. Reductive alkylation using NaCNBH₃ is carried out at medium to low pH with limited excess of the aldehyde resulting in alkylation taking place at the N-terminal. Example of a related pegylation is provided by Baker et al (Bioconjugate Chem. 17, 179-188 (2006)). In the successive step, TGase-mediated enzymatic reaction results in the modification of Gln at position 141. Conjugation of hGH with GHBP occurs via reductive alkylation. Reductive alkylation is exemplified herein and is well-recognized in the art.

In one embodiment, periodate-mediated oxidation of Ser-GHBP (GHBP extended at the N-terminus with serine) is performed, resulting in a compound for conjugation with an aniline in the reductive amination and the reaction is depicted as follows:

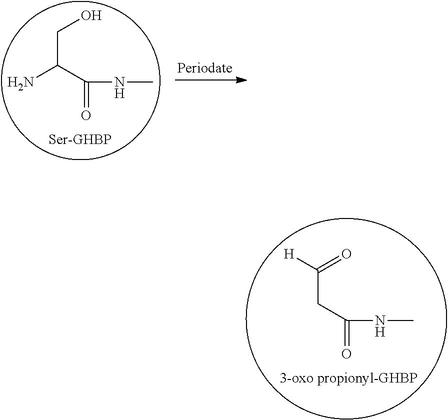

The resulting compound 3-oxo propionyl-GHBP is reacted with transglutaminase-modified hGH in the presence of a suitable reducing agent, for ex: NaCNBH₃ to yield hGH-GHBP of the formula (VI) as shown below:

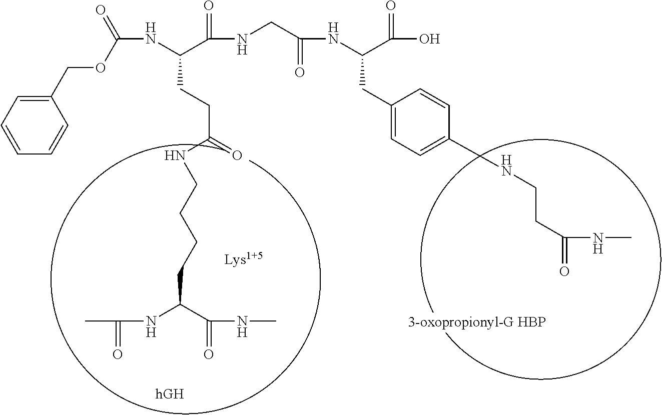

### Chemistry III

In one embodiment, the conjugate hGH-GHBP is prepared as illustrated below:

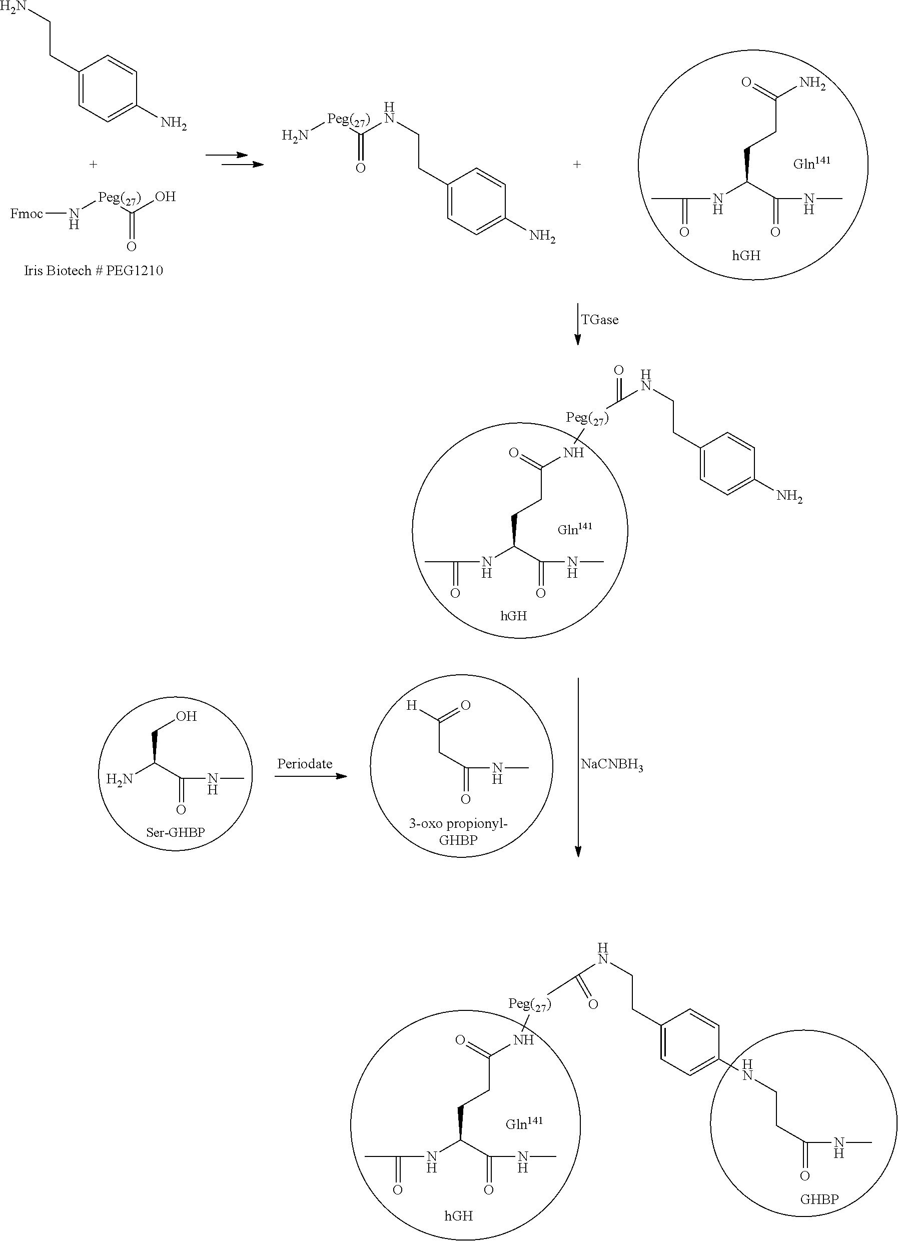

The derivatization process as shown above provides a PEG linker attached to hGH in position Gln-141. GHBP extended at the N-terminus by serine as described elsewhere herein provides a modified GHBP that may be conjugated to TGase-modified hGH to form hGH-GHBP.

### Chemistry IV

In one embodiment, the conjugate hGH-GHBP is prepared as illustrated below:

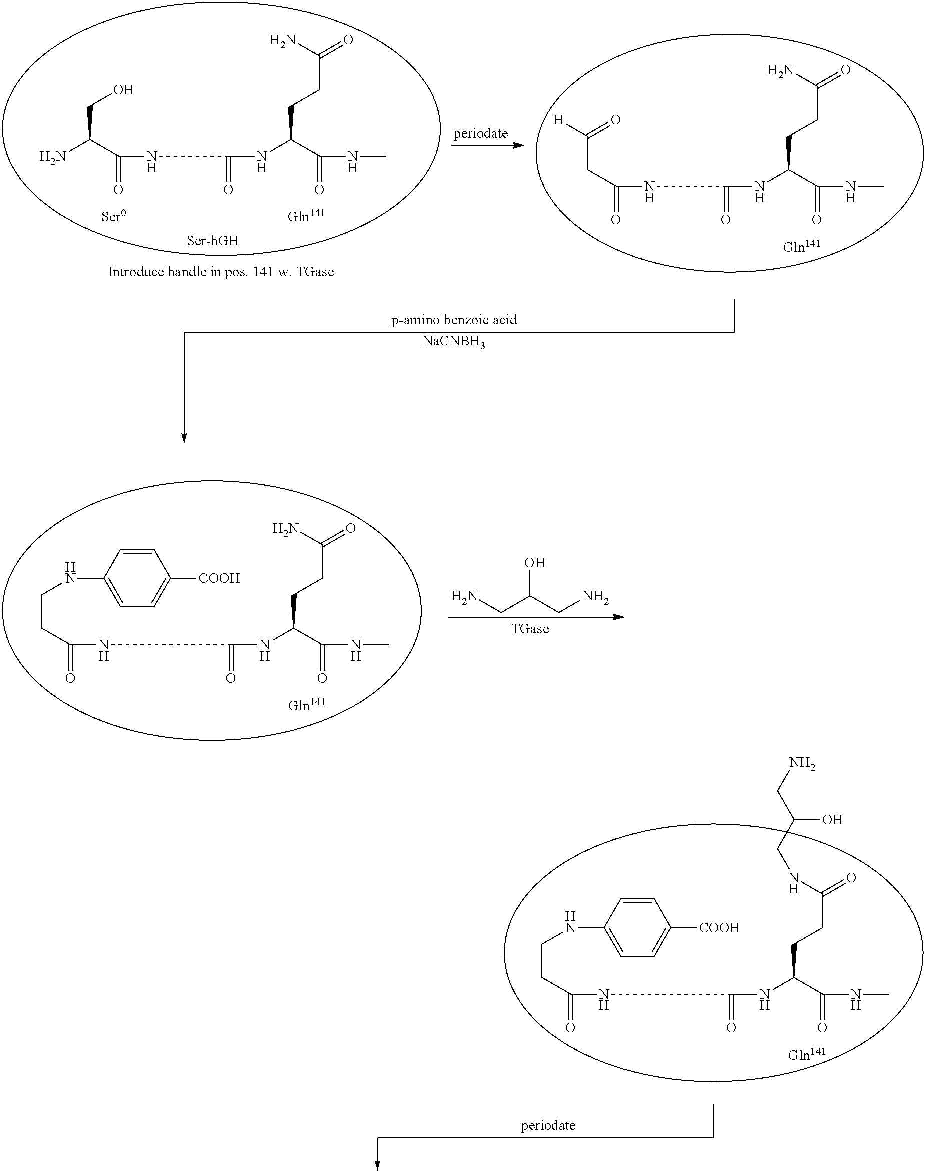

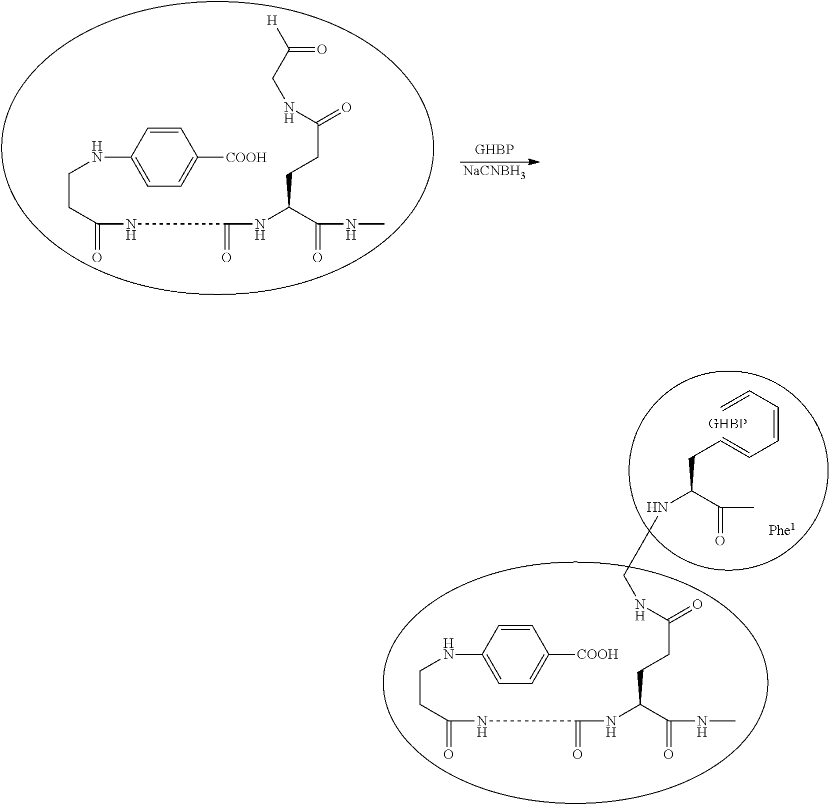

In the first two steps of chemistry IV a blocking of the N-terminal of growth hormone is introduced by first oxidizing the starting compound to the aldehyde which subsequently is reacted with an aniline in a reductive amination reaction. This blocking is introduced to increase yield and purity of the final compound. In the third step a handle is introduced site selectively in the glutamine in the position corresponding to position 141 in hGH using a transglutaminase catalyzed transamination reaction. In the fourth step this handle is converted into an aldehyde which finally in the fifth step is conjugated to the N-terminal of the GHBP compound.

### General Chemistry—Reducing Agent

NaCNBH₃ is mentioned herein as an example of a suitable reducing agent. However, a number of other reagents may be considered as alternatives to NaCNBH₃ as reducing agent for the conversion of imines into secondary amines. Thus, imines derived from oxidized carbohydrates and proteins have been reduced in aqueous solution with the commercially available adduct of borane (BH₃) and pyridine (Hashimoto et al., J. Biochem. 123, 468-478 (1998); Yoshida and Lee, Carbohydr. Res 251, 175-186 (1994)). Related reagents are adducts of borane and dimethylsulfide, phosphines, phosphites, substituted pyridines, pyrimidines, imidazoles, pyrazoles, thiazoles, sulfides, ethers, and the like. Further alternatives to NaCNBH₃ are catalytic hydrogenation (heterogeneous or homogeneous), NaBH₄, (Ehrenfreund-Kleinmann et al., Biomaterials 23, 1327-1335 (2002); Zito and Martinez-Carrion, J. Biol. Chem. 255, 8645-8649 (1980)), NaCNHB(OAc)₃ (Drummond et al., Proteomics 1, 304-310 (2001)), NADPH (the reduced form of NADP, nicotineamide adenine dinucleotide phosphate) or related dihydropyridines (Itoh et al., Tetrahedron Lett. 43, 3105-3108 (2002)), or mixtures of NaBH₄ and AcOH. NADPH may also be used in combination with a suitable enzyme, such as glutamate dehydrogenase (Fisher et al., J. Biol. Chem. 257, 13208-13210 (1982)). Each of these reagents may require adjustment of the pH of the solution, in order to provide for a high rate of imine-reduction if compared to the rate of hydrogen-formation (hydronium ion reduction). Thus, some reagents may reduce the imine under neutral or basic reaction conditions, whereas other reagents may require more acidic reaction conditions. Furthermore, some of these reagents may require the use of cosolvents, such as formamide, NMP, acetonitrile, ethylene glycol, isopropanol, and the like, in order to improve the solubility of all reactants or in order to reduce the concentration of water, and thus the rate of hydronium ion reduction. In one embodiment NaCNBH₃ is used as the reducing agent.

The invention relates to growth hormone compound conjugates having improved pharmacological properties, wherein the improved pharmacological properties is in particular intended to indicate for instance an increase in the functional in vivo half-life, the plasma in vivo half-life, the mean residence time, a decrease in the renal clearance or reduction in immunogenicity.

The term “functional in vivo half-life” is used in its normal meaning, i.e., the time at which 50% of the biological activity of the peptide, for instance growth hormone, or conjugated peptide, for instance growth hormone, is still present in the body/target organ, or the time at which the activity of the peptide, for instance growth hormone, or peptide, for instance growth hormone, conjugate is 50% of its initial value. As an alternative to determining functional in vivo half-life, “in vivo plasma half-life” may be determined, i.e., the time at which 50% of the peptide, for instance growth hormone, or peptide, for instance growth hormone, conjugate circulate in the plasma or bloodstream prior to being cleared. Determination of plasma half-life is often more simple than determining functional half-life and the magnitude of plasma half-life is usually a good indication of the magnitude of functional in vivo half-life. Alternative terms to plasma half-life include serum half-life, circulating half-life, circulatory half-life, serum clearance, plasma clearance, and clearance half-life.

Compounds of the present invention exert growth hormone activity and may be used in the treatment of diseases or states which will benefit from an increase in the amount of circulating growth hormone. Such diseases or states include growth hormone deficiency (GHD); Turner Syndrome; Prader-Willi syndrome (PWS); Noonan syndrome; Down syndrome; chronic renal disease, juvenile rheumatoid arthritis; cystic fibrosis, HIV-infection in children receiving HAART treatment (HIV/HALS children); short children born short for gestational age (SGA); short stature in children born with very low birth weight (VLBW) but SGA; skeletal dysplasia; hypochondroplasia; achondroplasia; idiopathic short stature (ISS); GHD in adults; fractures in or of long bones, such as tibia, fibula, femur, humerus, radius, ulna, clavicula, matacarpea, matatarsea, and digit; fractures in or of spongious bones, such as the scull, base of hand, and base of food; patients after tendon or ligament surgery in e.g. hand, knee, or shoulder; patients having or going through distraction oteogenesis; patients after hip or discus replacement, meniscus repair, spinal fusions or prosthesis fixation, such as in the knee, hip, shoulder, elbow, wrist or jaw; patients into which osteosynthesis material, such as nails, screws and plates, have been fixed; patients with non-union or mal-union of fractures; patients after osteatomia, e.g. from tibia or 1st toe; patients after graft implantation; articular cartilage degeneration in knee caused by trauma or arthritis; osteoporosis in patients with Turner syndrome; osteoporosis in men; adult patients in chronic dialysis (APCD); malnutritional associated cardiovascular disease in APCD; reversal of cachexia in APCD; cancer in APCD; chronic abstractive pulmonal disease in APCD; HIV in APCD; elderly with APCD; chronic liver disease in APCD, fatigue syndrome in APCD; Chron's disease; impaired liver function; males with HIV infections; short bowel syndrome; central obesity; HIV-associated lipodystrophy syndrome (HALS); male infertility; patients after major elective surgery, alcohol/drug detoxification or neurological trauma; aging; frail elderly; osteo-arthritis; traumatically damaged cartilage; erectile dysfunction; fibromyalgia; memory disorders; depression; traumatic brain injury; subarachnoid haemorrhage; very low birth weight; metabolic syndrome; glucocorticoid myopathy; or short stature due to glucocorticoid treatment in children. Growth hormone compound conjugate according to the invention may also be used for acceleration of the healing of muscle tissue, nervous tissue or wounds; the acceleration or improvement of blood flow to damaged tissue; or the decrease of infection, rate in damaged tissue. The present invention thus provides a method for treating these diseases or states, the method comprising administering to a patient in need thereof a therapeutically effective amount of a growth hormone compound conjugate according to the present invention.

A “therapeutically effective amount” of a compound according to the invention as used herein means an amount sufficient to cure, alleviate or partially arrest the clinical manifestations of a given disease and its complications. An amount adequate to accomplish this is defined as “therapeutically effective amount”. Effective amounts for each purpose will depend on e.g. the severity of the disease or injury as well as the weight, sex, age and general state of the subject. It will be understood that determining an appropriate dosage may be achieved using routine experimentation, by constructing a matrix of values and testing different points in the matrix, which is all within the ordinary skills of a trained physician or veterinary.

Typically, the amount of derivatized growth hormone administered is in the range from 10⁻⁷-10⁻³ g GH/kg body weight, such as 10⁻⁶-10⁻⁴ g GH/kg body weight, such as 10⁻⁵-10⁻⁴ g GH/kg body weight, wherein g GH designates the weight of the GH peptide without the GHBP.

In one embodiment, the invention provides the use of a growth hormone compound conjugate according to the invention in the manufacture of a medicament used in the treatment of the above mentioned diseases or states.

The present invention is also directed to pharmaceutical compositions comprising a growth hormone compound conjugate according to the invention. In one aspect, such a pharmaceutical composition comprises a growth hormone compound conjugate according to the invention, which is present in a concentration from 10-15 mg/ml to 200 mg/ml, such as e.g. 10-10 mg/ml to 5 mg/ml and wherein said composition has a pH from 2.0 to 10.0. The composition may further comprise a buffer system, preservative(s), tonicity agent(s), chelating agent(s), stabilizers and surfactants. In one embodiment of the invention the pharmaceutical composition is an aqueous composition, i.e. composition comprising water. Such composition is typically a solution or a suspension. In one embodiment of the invention the pharmaceutical composition is an aqueous solution. The term “aqueous composition” is defined as a composition comprising at least 50% w/w water. Likewise, the term “aqueous solution” is defined as a solution comprising at least 50% w/w water, and the term “aqueous suspension” is defined as a suspension comprising at least 50% w/w water.

In one embodiment the pharmaceutical composition is a freeze-dried composition, whereto the physician or the patient adds solvents and/or diluents prior to use.

In one embodiment the pharmaceutical composition is a dried composition (e.g. freeze-dried or spray-dried) ready for use without any prior dissolution.

In one embodiment the invention relates to a pharmaceutical composition comprising an aqueous solution of a growth hormone compound conjugate according to the invention, and a buffer, wherein said growth hormone compound conjugate according to the inventionmay be present in a concentration from 0.1-100 mg/ml or above, and wherein said composition has a pH from about 2.0 to about 10.0, and wherein mg GH designates the weight of the GH peptide without the GHBP.

In one embodiment of the invention the pH of the composition is selected from the list consisting of 2.0, through 10.0 with an upward gradation of 0.1, for ex. 2.1, 2.2. 2.3 and so on.

In one embodiment of the invention the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative embodiment of the invention.

In one embodiment of the invention the composition further comprises a pharmaceutically acceptable preservative. In one embodiment of the invention the preservative is selected from the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) or mixtures thereof. In one embodiment of the invention the preservative is present in a concentration from 0.1 mg/ml to 20 mg/ml. In one embodiment of the invention the preservative is present in a concentration from 0.1 mg/ml to 5 mg/ml. In one embodiment of the invention the preservative is present in a concentration from 5 mg/ml to 10 mg/ml. In one embodiment of the invention the preservative is present in a concentration from 10 mg/ml to 20 mg/ml. Each one of these specific preservatives constitutes an alternative embodiment of the invention. The use of a preservative in pharmaceutical compositions is well-known to the skilled person. For convenience reference is made to Remington: The Science and Practice of Pharmacy, 20^{th} edition, 2000.

In one embodiment of the invention the composition further comprises an isotonic agent. In one embodiment of the invention the isotonic agent is selected from the group consisting of a salt (e.g. sodium chloride), a sugar or sugar alcohol, an amino acid (e.g. glycine, histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol) polyethyleneglycol (e.g. PEG400), or mixtures thereof. Any sugar such as mono-, di-, or polysaccharides, or water-soluble glucans, including for example fructose, glucose, mannose, sorbose, xylose, maltose, lactose, sucrose, trehalose, dextran, pullulan, dextrin, cyclodextrin, soluble starch, hydroxyethyl starch and carboxymethylcellulose-Na may be used. In one embodiment the sugar additive is sucrose. Sugar alcohol is defined as a C4-C8 hydrocarbon having at least one —OH group and includes, for example, mannitol, sorbitol, inositol, galactitol, dulcitol, xylitol, and arabitol. In one embodiment the sugar alcohol additive is mannitol. The sugars or sugar alcohols mentioned above may be used individually or in combination. There is no fixed limit to the amount used, as long as the sugar or sugar alcohol is soluble in the liquid preparation and does not adversely effect the stabilizing effects obtained using the methods of the invention. In one embodiment, the sugar or sugar alcohol concentration is between about 1 mg/ml and about 150 mg/ml. In one embodiment of the invention the isotonic agent is present in a concentration from 1 mg/ml to 50 mg/ml. In one embodiment of the invention the isotonic agent is present in a concentration from 1 mg/ml to 7 mg/ml. In one embodiment of the invention the isotonic agent is present in a concentration from 8 mg/ml to 24 mg/ml. In one embodiment of the invention the isotonic agent is present in a concentration from 25 mg/ml to 50 mg/ml. Each one of these specific isotonic agents constitutes an alternative embodiment of the invention. The use of an isotonic agent in pharmaceutical compositions is well-known to the skilled person. For convenience reference is made to Remington: The Science and Practice of Pharmacy, 20th edition, 2000.

The invention is further illustrated by, but not limited to, the following embodiments.
1. A compound having the formula:

A-B-C
A is a radical of a growth hormone compound;
B is a linking and spacing bivalent residue;
C is a radical of a growth hormone binding protein compound; and
- is a covalent bond,
   wherein the linkages between A and B and/or the linkage between B and C are not provided by recombinant expression of a nucleic acid comprising a nucleic acid sequence encoding a fusion protein having the structure A-B-C.
2. A compound according to embodiment 1, wherein B is not a pure peptide chain.
3. A compound having the formula:

A-B-C
A is a radical of a growth hormone compound;
B is a linking and spacing bivalent residue;
C is a radical of a growth hormone binding protein compound; and
- is a covalent bond,
   wherein B is not a pure peptide chain.
4. A compound according to any of embodiments 1 to 3, wherein the growth hormone compound comprises an amino acid sequence, which has at least 80% identity to SEQ ID No. 1 and which growth hormone compound has an activity in the assay described in Example 7 of at least 10% of hGH.
5. A compound according to embodiment 4, wherein the growth hormone compound comprises an amino acid sequence having at least 85%, such as at least 90%, for instance at least 95%, such as at least 96%, for instance at least 97%, such as at least 98%, for instance at least 99% identity to SEQ ID No. 1.
6. A compound according to embodiment 4 or embodiment 5, wherein the activity of the growth hormone compound is at least 20%, such as at least 30%, for instance at least 40%, such as at least 50%, for instance at least 60%, such as at least 70%, for instance at least 80%, such as at least 90% of hGH, for instance substantially the same activity as hGH.
7. A compound according to any of embodiments 1 to 6, wherein the growth hormone compound is a growth hormone analogue as described in WO2006048777, WO2004022593, WO2005018659, WO2005074524; WO2005074546, WO2005074650, U.S. Pat. No. 5,849,535, U.S. Pat. No. 6,136,563, U.S. Pat. No. 6,022,711, or U.S. Pat. No. 6,143,523.
8. A compound according to any of embodiments 1 to 6, wherein the growth hormone compound is hGH.
9. A compound according to any of embodiments 1 to 8, wherein the growth hormone binding protein compound comprises an amino acid sequence, which has at least 80% identity to SEQ ID No. 2 and which peptide is capable of binding to growth hormone with a KD<100 nM.
10. A compound according to embodiment 9, wherein the growth hormone binding protein compound comprises an amino acid sequence, which has at least 85%, such as at least 90%, for instance at least 95%, such as at least 96%, for instance at least 97%, such as at least 98%, for instance at least 99% identity to SEQ ID No. 2.
11. A compound according to embodiment 10, wherein the growth hormone binding protein compound comprises the amino acid sequence of SEQ ID No. 2.
12. A compound according to any of embodiments 1 to 8, wherein the growth hormone binding protein compound comprises a fragment of a peptide comprising an amino acid sequence, which amino acid sequence has at least 80%, for instance at least 85%, such as at least 90%, for instance at least 95%, such as at least 96%, for instance at least 97%, such as at least 98%, for instance at least 99%, such as 100% identity to SEQ ID No. 2, which fragment has retained a significant amount of the GHBP activity of such a peptide.
13. A compound according to any of embodiments 1 to 12, wherein at least one of the covalent bonds A-C or B-C is established by an enzyme.
14. A compound according to embodiment 13, wherein said enzyme is selected from the group consisting of transglutaminases, serine proteases and cysteine proteases.
15. A compound according to embodiment 14, wherein said enzyme is a transglutaminase selected from the group consisting of microbial transglutaminase, tissue transglutaminase and factor XIII.
16. A compound according to embodiment 15, wherein the microbial transglutaminase is a transglutaminase from S. mobaraense or S. ladakanum.
17. A compound according to embodiment 15, wherein the microbial transglutaminase is a transglutaminase as described in WO2007/02029, WO2008/020075 and/or WO2008/020075.
18. A compound according to any of embodiments 1 to 17, wherein B comprises at least 10 covalent bonds.
19. A compound according to embodiment 18, wherein B comprises at least 30 covalent bonds.
20. A compound according to embodiment 19, wherein B comprises at least 80 covalent bonds.
21. A compound according to embodiment 20, wherein B comprises at least 300 covalent bonds.
22. A compound according to embodiment 21, wherein B comprises at least 1000 covalent bonds.
23. A compound according to embodiment 22, wherein B comprises at least 3000 covalent bonds.
24. A compound according to any of embodiments 1 to 23, wherein B comprises a PEG unit.
25. A compound according to embodiment 24, wherein B comprises the structure

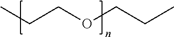

wherein n is an integer larger than 1, and the molecular weight of the structure is between around 100 Da to around 1,000,000 kDa.
26. A compound according to embodiment 24 or embodiment 25, wherein B comprises a PEG unit of from around 300 to around 50,000 kDa.
27. A compound according to embodiment 26, wherein B comprises a PEG unit with a molecular weight of for instance around 300, 500, 750, 1,000, 2,000, 5,000, 10,000, 20,000, 30,000, 40,000 or 50,000 kDa
28. A compound according to embodiment 26, wherein B comprises a PEG unit with a molecular weight of from around 1,000 to 10,000.
29. A compound according to any of embodiments 1 to 3, wherein B is selected from the group consisting of . . .

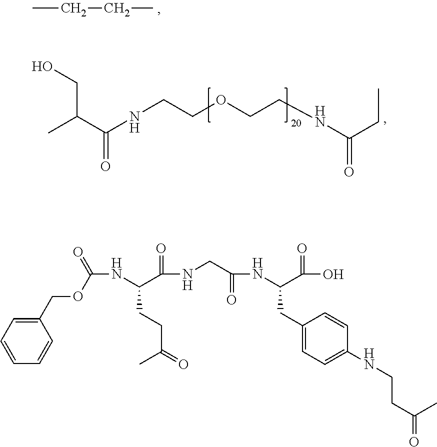

30. A compound according to any of embodiments 15 to 17 of the formula

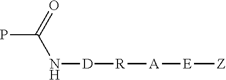

wherein P—C(O)—NH— represents the growth hormone compound radical obtained by removing a hydrogen from —NH₂ in the side chain of Gln;
D represents a bond or oxygen;
R represents a linker or a bond;
E represents a linker or a bond;
A represents an oxime, hydrazone, phenylhydrazone, semicarbazone, triazole or isooxazolidine moiety; and
Z is a radical of a growth hormone binding protein compound as described above.
31. A compound according to embodiment 30, wherein the growth hormone binding protein compound comprises an amino acid sequence, which has at least 80% identity to SEQ ID No. 2 and which peptide is capable of binding to growth hormone with a KD<100 nM.
32. A compound according to embodiment 31, wherein the growth hormone binding protein compound comprises an amino acid sequence, which has at least 85%, such as at least 90%, for instance at least 95%, such as at least 96%, for instance at least 97%, such as at least 98%, for instance at least 99% identity to SEQ ID No. 2.
33. A compound according to embodiment 32, wherein the growth hormone binding protein compound comprises the amino acid sequence of SEQ ID No. 2.
34. A compound according to embodiment 30, wherein the growth hormone binding protein compound comprises a fragment of a peptide comprising an amino acid sequence, which amino acid sequence has at least 80%, for instance at least 85%, such as at least 90%, for instance at least 95%, such as at least 96%, for instance at least 97%, such as at least 98%, for instance at least 99%, such as 100% identity to SEQ ID No. 2, which fragment has retained a significant amount of the GHBP activity of such a peptide.
35. A compound according to any of embodiments 30 to 34, wherein A represents an oxime or triazole moiety.
36. A compound according to any of embodiments 30 to 35, wherein the growth hormone compound is conjugated in the position corresponding to position 141 in SEQ ID No. 1.
37. A compound according to any of embodiments 15 to 17 of the forumla (I)

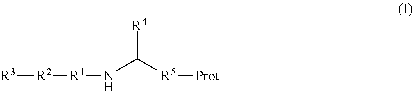

wherein
Prot represents a radical of the growth hormone compound as described above, wherein said radical is formally generated by the formal removal of a hydrogen atom from an amino group (—NH₂) of said growth hormone compound,
R¹ represents arylene or a heteroarylene, optionally substituted with a C₁₆-alkyl, halogen, cyano, nitro, hydroxyl, carboxyl, or aryl group;
R² represents a bond or a linker, wherein said linker comprises a diradical selected from the group consisting of C(═O)NH, NH, O, S, OP(O)(OH)O, OC(═O)NH, NHC(═O)NH, (CH₂)₁₁₀, O(CH₂)₃NHC(═O), C(═O)NH(CH₂)₂₃₀, (CH₂)₁₃₀C(═O)NH(CH₂)₂₃₀, (CH₂)₀₃₀C(═O)NH(CH₂CH₂O)₁₁₀(CH₂)₁₅C(═O), C(═O)NH[(CH₂CH₂O)₁₁₀(CH₂)₁₅C(═O)]₁₅NH(CH₂)₂₃₀, C(═O), (CH₂)₁₂₀—NHC(═O), (CH₂)₁₃₀C(═O), NHC(═O)NH(CH₂)₂₃₀, (CH₂)₁₃₀—NHC(═O)NH(CH₂)₂₃₀, (CH₂)₀₃₀C(═O)NH(CH₂)₂₃₀NHC(═O)(CH₂)₀₃₀, (CH₂)₀₃₀C(═O)NH(CH₂CH₂O)₁₃₀CH₂CH₂NHC(═O)(CH₂)₀₃₀, or NH(CH₂)₂₃₀,

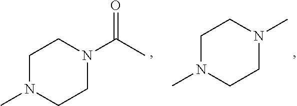

and combinations thereof, and
R³ represents the radical of the growth hormone binding protein compound as described above;
R⁴ represents hydrogen or C₁₆-alkyl; and
R⁵ represents —CH₂— or —C(═O)—.
38. A compound according to embodiment 37, wherein R¹ represents arylene or a heteroarylene, optionally substituted with a C₁₆-alkyl, halogen, cyano, nitro, hydroxyl, carboxyl, or C₅₋₂₂-aryl group.
39. A compound according to embodiment 38, wherein R¹ represents arylene or a heteroarylene, optionally substituted with a C₁₆-alkyl, halogen, cyano, nitro, hydroxyl, carboxyl, or C₆₋₁₈-aryl group.
40. A compound according to embodiment 39, wherein R¹ represents arylene or a heteroarylene, optionally substituted with a C₁₆-alkyl, halogen, cyano, nitro, hydroxyl, carboxyl, or C₄₋₁₆-aryl group.
41. A compound according to embodiment 40, wherein R¹ represents arylene or a heteroarylene, optionally substituted with a C₁₆-alkyl, halogen, cyano, nitro, hydroxyl, carboxyl, or C₆₋₈-aryl group.
42. A compound according to any of embodiments 37 to 41, wherein R¹ represents a C₅₋₂₂-arylene, optionally substituted as described above.
43. A compound according to embodiment 42, wherein R¹ represents a C₆₋₁₈-arylene, optionally substituted as described above.
44. A compound according to embodiment 43, wherein R¹ represents a C₆₋₁₄-arylene, optionally substituted as described above.
45. A compound according to any of embodiments 37 to 41, wherein R¹ represents a C₅₋₂₂-heteroarylene, optionally substituted as described above.
46. A compound according to embodiment 45, wherein R¹ represents a C₆₋₁₈-heteroarylene, optionally substituted as described above.
47. A compound according to embodiment 46, wherein R¹ represents a C₆₋₁₄-heteroarylene, optionally substituted as described above.
48. A compound according to any of embodiments 37 to 41, wherein R¹ represents a C₆₋₈-arylene, a C₁₂₋₁₈-arylene or a C₅₋₁₈-heteroarylene, optionally substituted as described above.
49. A compound according to embodiment 48, wherein R¹ represents a phenylene or a pyridylene group.
50. A compound according to embodiment 49, wherein R¹ represents 1,4-phenylene.
51. A compound according to any of embodiments 37 to 50, in which R² represents a bond, —C(═O)—, C(═O)NH, or

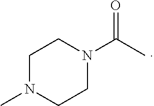

52. A compound according to embodiment 51, wherein R² represents C(═O)NH.
53. A compound according to any of embodiments 37 to 52, in which R⁴ represents hydrogen.
54. A compound according to any of embodiments 37 to 52, in which R⁴ represents C₁₋₆-alkyl.
55. A compound according to any of embodiments 37 to 54, in which R⁵ represents —CH₂—.
56. A compound according to any of embodiments 37 to 54, in which R⁵ represents —C(═O)—.
57. A compound according to any of embodiments 37 to 56, wherein Prot is obtainable from a growth hormone compound-derived aldehyde or ketone of formula

R⁶—C(═O)—R⁵-Prot,

wherein
R⁵ is as described above, and
R⁶ represents hydrogen or an optionally substituted α-carbon atom.
58. A compound according to embodiment 57, wherein R⁶ represents hydrogen, C₁₆-alkyl or C₁₆-cycloalkyl.
59. A compound according to embodiment 58, wherein R⁶ represents hydrogen, methyl, ethyl, propyl, cyclopropyl, cyclobutyl, cyclopentyl, or cyclohexyl.
60. A compound according to embodiment 59, wherein R⁶ represents hydrogen or methyl.
61. A compound according to any of embodiments 37 to 60, wherein Prot represents a radical of a growth hormone compound as described above, wherein said radical is formally generated by the removal of a hydrogen atom from an amino group (—NH₂) of said growth hormone compound, and wherein said amino group is the N-terminal amino group of the peptide, a side-chain amino group of lysine residue in the peptide, or a side-chain amino group of glutamine or asparagine (C(═O)NH₂) residue in the peptide.
62. A compound according to embodiment 61, wherein Prot represents a radical of a growth hormone compound formally generated by removal of one hydrogen atom from the N-terminal amino group.
63. A compound according to embodiment 61, wherein Prot represents a radical of a growth hormone compound formally generated by removal of one hydrogen atom from a side-chain amino group of lysine residue in the peptide.
64. A compound according to embodiment 61, wherein Prot represents a radical of a growth hormone compound formally generated by removal of one hydrogen atom from a side-chain amino group of glutamine or asparagine residue in the peptide.
65. A compound according to any of embodiments 37 to 64, wherein the Prot radical represents a peptide radical formally generated by removal of one hydrogen atom from the side-chain aminocarbonyl group (H₂N—CO—) of the glutamine at the position corresponding to position 40 in SEQ ID No. 1.
66. A compound according to any of embodiments 37 to 64, wherein the Prot radical represents a peptide radical formally generated by removal of one hydrogen atom from the side-chain aminocarbonyl group (H₂N—CO—) of the glutamine at the position corresponding to position 141 in SEQ ID No. 1.
67. A compound according to embodiment 14, wherein said enzyme is a serine protease selected from the group consisting of carboxypeptidase Y, trypsin and chymotrypsin.
68. A compound according to embodiment 14, wherein said enzyme is a cysteine protease selected from the group consisting of papain, sortase A and sortase B.
69. A compound according to any of embodiments 1 to 68, wherein at least one of the covalent bonds A-C or B-C is a thioether bond to a cysteine residue.
70. A pharmaceutical composition comprising a compound according to any of embodiments 1 to 69 and a pharmaceutically acceptable carrier.
71. A method of treating a disease state in a mammal that will benefit from increase in the activity of growth hormone comprising administering to the mammal an effective amount of a pharmaceutical composition according to embodiment 70.
72. A compound according to any of embodiments 1 to 69 for therapy.
73. A compound according to any of embodiments 1 to 69 for treatment of disease states in mammal that will benefit from increase in the activity of growth hormone.
74. Use of a compound according to any of embodiments 1 to 69 for the preparation of a pharmaceutical composition for treating disease states in a mammal that will benefit from increase in the activity of growth hormone.
75. A method for preparing a compound according to any of embodiments 1 to 69, said method comprising
(a) enzymatic derivitization of the growth hormone compound, and
(b) conjugating the enzymatically derivatized growth hormone compound from step (a) to the growth hormone binding protein compound.
76. A method for preparing a compound according to any of embodiments 30 to 36, wherein said method comprising the steps of
i) reacting in one or more steps the growth hormone compound as described above with a first compound comprising one or more functional groups or latent functional groups, which are not accessible in any of the amino acids residues constituting said growth hormone compound, in the presence of transglutaminase capable of catalysing the incorporation of said first compound into said growth hormone compound to form a functionalised growth hormone compound; and
ii) optionally activate the latent functional group; and
iii) reacting in one or more steps said functionalised growth hormone compound with a second compound comprising one or more functional groups, wherein said functional group(s) do not react with functional groups accessible in the amino acid residues constituting said peptide, and wherein said functional group(s) in said second compound is capable of reacting with said functional group(s) in said first compound so that a covalent bond between said functionalised peptide and said second compound is formed, and wherein said second compound comprises the radical of the growth hormone binding protein compound as described above.
77. A method according to embodiment 76, wherein a Gln-residue containing growth hormone compound represented by the formula

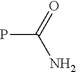

is reacted in one or more steps with a nitrogen containing nucleophile (first compound) represented by the formula

H₂N-D-R—X

in the presence of a transglutaminase enzyme to form a transaminated peptide of the formula

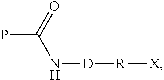

optionally the latent functional group comprised in Xis activiated,
said transaminated growth hormone compound being further reacted with a second compound of the formula

Y-E-Z

to form a conjugated growth hormone compound of the formula

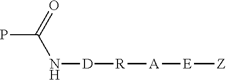

wherein D represents a bond or oxygen;
R represents a linker or a bond;
X represents a radical comprising a functional group or a latent functional group not accessible in the amino acid residues constituting the peptide P—C(O)—NH₂;
Y represents a radical comprising one or more functional groups which groups react with functional groups present in X, and which functional groups do not react with functional groups accessible in the peptide P—C(O)—NH₂;
E represents a linker or a bond;
A represents the moiety formed by the reaction between the functional groups comprised in X and Y; and
Z comprises the radical of the growth hormone binding protein compound as described above.
78. A method according to embodiment 77, wherein A represents an oxime, hydrazone, phenylhydrazone, semicarbazone, triazole or isooxazolidine moiety.
79. A method according to embodiment 77 or embodiment 78, wherein the functional group or latent functionnal group comprised in X is selected from or can be activated to keto-, aldehyde-, —NH—NH2, —O—C(O)—NH—NH2, —NH—C(O)—NH—NH2, —NH—C(S)—NH—NH2, —NHC(O)—NH—NH—C(O)—NH—NH2, —NH—NH—C(O)—NH—NH2, —NH—NH—C(S)—NH—NH2, —NH—C(O)—C6H4-NH—NH2, —C(O)—NH—NH2, —O—NH2, —C(O)—O—NH2, —NH—C(O)—O—NH2, —NH—C(S)—O—NH2, alkyne, azide or nitril-oxide.
80. A method according to any of embodiments 77 to 79, wherein the functional group present in Y is selected from amongst keto-, aldehyde-, —NH—NH2, —O—C(O)—NH—NH2, —NH—C(O)—NH—NH2, —NH—C(S)—NH—NH2, —NHC(O)—NH—NH—C(O)—NH—NH2, —NH—NH—C(O)—NH—NH2, —NH—NH—C(S)—NH—NH2, —NH—C(O)—C6H4-NH—NH2, —C(O)—NH—NH2, —O—NH2, —C(O)—O—NH2, —NH—C(O)—O—NH2, —NH—C(S)—O—NH2, alkyne, azide and nitril-oxide.
81. A method according to any of embodiments 77 to 80, wherein X is selected from or can be activated to keto- or aldehyde-derivatives, and Y is selected from —NH—NH₂, —O—C(O)—NH—NH₂, —NH—C(O)—NH—NH₂, —NH—C(S)—NH—NH₂, —NHC(O)—NH—NH—C(O)—NH—NH₂, —NH—NH—C(O)—NH—NH₂, —NH—NH—C(S)—NH—NH₂, —NH—C(O)—C₆H₄—NH—NH₂, —C(O)—NH—NH₂, —O—NH₂, —C(O)—O—NH₂, —NH—C(O)—O—NH₂, and —NH—C(S)—O—NH₂.
82. A method according to embodiment 81, wherein the latent group comprised in X is selected amongst

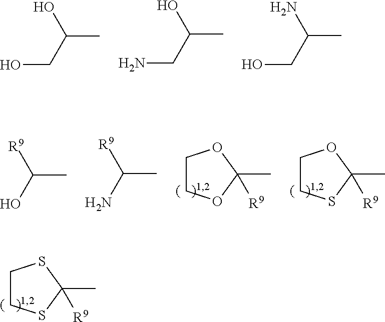

wherein R⁹ is selected amongst H, C₁₋₆alkyl, aryl and heteroaryl.
83. A method according to any of embodiments 77 to 80, wherein X and Y each represent a different member of the group consisting of alkyne and triazole, or of the group consisting of alkyne and nitril-oxide.
84. A method according to any of embodiments 77 or 81, wherein said nitrogen containing nucleophile is selected from 4-(aminomethyl)phenyl ethanone, 4-(2-aminoethyl)phenyl ethanone, N-(4-acetylphenyl) 2-aminoacetamide, 1-[4-(2-aminoethoxy)phenyl]ethanone, 1-[3-(2-aminoethoxy)phenyl]ethanone, 1,4-bis(aminoxy)butane, 3-oxapentane-1,5-dioxyamine, 1,8-diaminoxy-3,6-dioxaoctane, 1,3-bis(aminoxy)propan-2-ol, 1,11-bis(aminoxy)-3,6,9-trioxaundecane, 1,3-diamino-2-propanol, 1,2-bis(aminoxy)ethane, and 1,3-bis(aminoxy)propane.
85. A method for preparing a compound according to any of embodiments to 37 to 66, said method comprising the steps of
(a) treatment of an aldehyde or ketone derived from the growth hormone compound with a property-modifying group-derived aniline or heteroarylamine to yield an imine or a hemiaminal, wherein said property-modifying group comprises a growth hormone binding protein or fragment thereof as described above,
(b) treatment of this imine or hemiaminal with a suitable reducing agent, such as NaCNBH₃, to yield a secondary amine.
86. A method according to embodiment 85, wherein the growth hormone compound-derived aldehyde or ketone is prepared by periodate-mediated oxidation of a peptide compound containing a 2aminoethanol substructure.
87. A method according to any of embodiments 85, wherein the growth hormone compound-derived aldehyde or ketone is prepared by hydrolysis of an acetal or a hemiacetal.
88. A method according to any of embodiments 85, wherein the growth hormone compound-derived aldehyde is prepared by periodate-mediated oxidation of a peptide containing serine or threonine as the N-terminal amino acid.
89. A method according to any of embodiments 85, wherein the growth hormone compound-derived aldehyde is prepared by periodate-mediated oxidation of a peptide transaminated enzymatically with 1,3diamino-2-propanol.
90. A method according to any of embodiments 85, wherein the growth hormone compound-derived aldehyde or ketone is prepared by feeding a genetically modified or unmodified organism producing said peptide with an unnatural amino acid containing an aldehyde or ketone functionality, wherein said amino acid will be incorporated into the peptide, followed by isolation and purification of the peptide into which the unnatural amino acid has been incorporated.
91. A method according to embodiment 90, wherein the unnatural amino acid is (acetylphenyl)alanine or (formylphenyl)alanine.
92. A method according to embodiment 90 or embodiment 91, wherein the mRNA encoding the peptide comprises at least one codon encoding phenylalanine.
93. A method according to any of embodiments 85 to 92, wherein the property-modifying group-derived aniline or heteroarylamine is of the formula

R³—R²—R¹—NH₂ (III)

wherein R¹, R², and R³ are as defined in embodiment 37.
94. A method according to embodiment 93, wherein R¹ represents arylene or a heteroarylene, optionally substituted with a C₁₆-alkyl, halogen, cyano, nitro, hydroxyl, carboxyl, or C₅₋₂₂-aryl group.
95. A method according to embodiment 94, wherein R¹ represents arylene or a heteroarylene, optionally substituted with a C₁₆-alkyl, halogen, cyano, nitro, hydroxyl, carboxyl, or C₆₋₁₈-aryl group.
96. A method according to embodiment 95, wherein R¹ represents arylene or a heteroarylene, optionally substituted with a C₁₆-alkyl, halogen, cyano, nitro, hydroxyl, carboxyl, or C₄₋₁₆-aryl group.
97. A method according to embodiment 96, wherein R¹ represents arylene or a heteroarylene, optionally substituted with a C₁₆-alkyl, halogen, cyano, nitro, hydroxyl, carboxyl, or C₆₋₈-aryl group.
98. A method according to any of embodiments 93 to 97, wherein R¹ represents a C₅₋₂₂-arylene, optionally substituted as described above.
99. A method according to embodiment 98, wherein R¹ represents a C₆₋₁₈-arylene, optionally substituted as described above.
100. A method according to embodiment 99, wherein R¹ represents a C₆₋₁₄-arylene, optionally substituted as described above.
101. A method according to any of embodiments 93 to 97, wherein R¹ represents a C₅₋₂₂-heteroarylene, optionally substituted as described above.
102. A method according to embodiment 101, wherein R¹ represents a C₆₋₁₈-heteroarylene, optionally substituted as described above.
103. A method according to embodiment 102, wherein R¹ represents a C₆₋₁₄-heteroarylene, optionally substituted as described above.
104. A method according to any of embodiments 93 to 97, wherein R¹ represents a C₆₋₈-arylene, a C₁₂₋₁₈-arylene or a C₅₋₁₈-heteroarylene, optionally substituted as described above.
105. A method according to embodiment 104, wherein R¹ represents a phenylene or a pyridylene group.
106. A method according to embodiment 105, in which R¹ represents 1,4-phenylene.
107. A method according to any of embodiments 93 to 106, wherein R² represents a bond, C(═O)NH, or

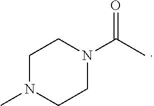

108. A method according to embodiment 107, wherein R² represents C(═O)NH.
109. A method according to any of embodiments 85 to 108, wherein the growth hormone compound-derived aldehyde or ketone is of the formula

R⁶—C(═O)—R⁵-Prot,

wherein
Prot is as described in embodiment 37,
R⁵ is —CH₂— or —C(═O)—, and
R⁶ represents hydrogen or an optionally substituted α-carbon atom.
110. A method according to embodiment 109, in which R⁵ represents —CH₂—.
111. A method according to embodiment 109, in which R⁵ represents —C(═O)—.
112. A method according to any of embodiments 109 to 111, wherein R⁶ represents hydrogen, C₁₆-alkyl or C₁₆-cycloalkyl.
113. A method according to embodiment 112, wherein R⁶ represents hydrogen, methyl, ethyl, propyl, cyclopropyl, cyclobutyl, cyclopentyl, or cyclohexyl.
114. A method according to embodiment 113, wherein R⁶ represents hydrogen or methyl.
115. A method according to any of embodiments 85 to 114, wherein the property-modifying group is conjugated to the peptide on the position corresponding to position 40 in SEQ ID No. 1.
116. A method according to embodiment 85 to 114, wherein the property-modifying group is conjugated to the peptide on the position corresponding to position 141 in SEQ ID No. 1.
117. A compound of formula (III)

R³—R²—R¹—NH₂ (III)

wherein R¹, R², and R³ are as defined in embodiment 37.
118. A compound according to embodiment 117, wherein R¹ represents arylene or a heteroarylene, optionally substituted with a C₁₆-alkyl, halogen, cyano, nitro, hydroxyl, carboxyl, or C₅₋₂₂-aryl group.
119. A compound according to embodiment 118, wherein R¹ represents arylene or a heteroarylene, optionally substituted with a C₁₆-alkyl, halogen, cyano, nitro, hydroxyl, carboxyl, or C₆₋₁₈-aryl group.
120. A compound according to embodiment 119, wherein R¹ represents arylene or a heteroarylene, optionally substituted with a C₁₆-alkyl, halogen, cyano, nitro, hydroxyl, carboxyl, or C₄₋₁₆-aryl group.
121. A compound according to embodiment 120, wherein R¹ represents arylene or a heteroarylene, optionally substituted with a C₁₆-alkyl, halogen, cyano, nitro, hydroxyl, carboxyl, or C₆₋₈-aryl group.
122. A compound according to any of embodiments 117 to 121, wherein R¹ represents a C₅₋₂₂-arylene, optionally substituted as described above.
123. A compound according to embodiment 122, wherein R¹ represents a C₆₋₁₈-arylene, optionally substituted as described above.
124. A compound according to embodiment 123, wherein R¹ represents a C₆₋₁₄-arylene, optionally substituted as described above.
125. A compound according to any of embodiments 117 to 121, wherein R¹ represents a C₅₋₂₂-heteroarylene, optionally substituted as described above.
126. A compound according to embodiment 125, wherein R¹ represents a C₆₋₁₈-heteroarylene, optionally substituted as described above.
127. A compound according to embodiment 126, wherein R¹ represents a C₆₋₁₄-heteroarylene, optionally substituted as described above.
128. A compound according to any of embodiments 117 to 121, wherein R¹ represents a C₆₋₈-arylene, a C₁₂₋₁₈-arylene or a C₅₋₁₈-heteroarylene, optionally substituted as described above.
129. A compound according to embodiment 128, wherein R¹ represents a phenylene or a pyridylene group.
130. A compound according to embodiment 129, wherein R¹ represents 1,4-phenylene.
131. A compound according to any of embodiments 117 to 130, in which R² represents a bond, —C(═O)—, C(═O)NH, or

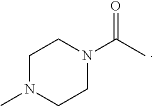

132. A compound according to embodiment 131, wherein R² represents C(═O)NH.
133. A method for preparing a compound according to any of embodiments 15 to 17, which method comprises
i) contacting the growth hormone compound as described above with a first compound having the following formula: R⁷GlnGlyR⁸, wherein R⁷ and R⁸ are groups suitable for further modification, in the presence of a transglutaminase, and
ii) reacting in one or more steps the product of step i) with a second compound comprising one or more functional groups, wherein said functional group(s) do not react with functional groups accessible in the amino acid residues constituting said peptide, and wherein said functional group(s) in said second compound is capable of reacting with R⁷ and/or R⁸, so that a covalent bond between the product of step i) and said second compound is formed, and wherein said second compound comprises the radical of the growth hormone binding protein compound as described above.
134. A method according to embodiment 133, wherein R⁷ contain an aromatic or heteroaromatic group.
135. A method according to embodiment 134, where R⁷ is a carbobenzyloxy group (PhCH₂OC(═O)).
136. A method according to any of embodiments 133 to 135, wherein R⁸ is a group containing a functional group selected from: CHO, ONH₂, ArNH₂, alkynyl, azide, NHNH₂, CHXCH₂Y or CHYCH₂X (where X is O or N, and Y is O), an acetal or a different latent aldehyde, SH, ZCH₂C═O (where Z is Cl, Br or I).
137. A method according to embodiment 133, wherein the first compound has the formula

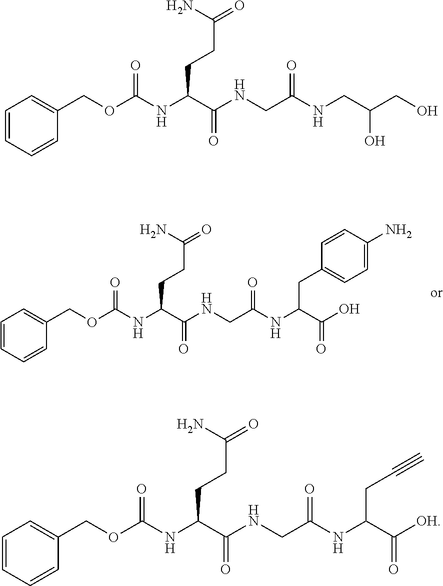

138. A method according to any of embodiments 133 to 136, wherein the growth hormone compound is conjugated at a lysine residue.
139. A method according to embodiment 138, wherein the growth hormone compound is conjugated in the position corresponding to position 145 in SEQ ID No. 1.

The present invention will be further illustrated in the following examples. However, it is to be understood that these examples are for illustrative purposes only, and should not be used to limit the scope of the present invention in any manner.

### EXAMPLES

The TGase used in the examples is microbial transglutaminase from Streptoverticiffium mobaraense according to U.S. Pat. No. 5,156,956.

The examples also contain the following general methods:

### Capillary Electrophoresis

Capillary electrophoresis was carried out using an Agilent Technologies 3DCE system (Agilent Technologies). Data acquisition and signal processing were performed using Agilent Technologies 3DCE ChemStation. The capillary was a 64.5 cm (56.0 cm efficient length) 50 μm i.d. “Extended Light Path Capillary” from Agilent. UV detection was performed at 200 nm (16 nm Bw,Reference 380 nm and 50 nm Bw). The running electrolyte was phosphate buffer 50 mM pH7 (method A). The capillary was conditioned with 0.1 M NaOH for 3 min, then with Milli-Q water for 2 min and with the electrolyte for 3 min. After each run, the capillary was flushed with milli-Q water for 2 min, then with phosphoric acid for 2 min, and with milli-Q water for 2 min. The hydrodynamic injection was done at 50 mbar for 4.0 s. The voltage was +25 kV. The capillary temperature was 30 C and the runtime was 10.5 min.

### Maldi-Tof Mass Spectrometry

Molecular weights were determined using the Autoflex Maldi-Tof instrument (Bruker). Samples were prepared using alfa-cyano-4-hydroxy-cinnamic acid as matrix.

### RP-HPLC

RP-HPLC analysis was performed on a Agilent 1100 system using a Vydac 218TP54 4.6 mm×250 mm 5 μm C-18 silica column (The Separations Group, Hesperia). Detection was by UV at 214 nm, 254 nm, 280 nm and 301 nm. The column was equilibrated with 0.1% trifluoracetic acid/H₂O and the sample was eluted by a suitable gradient of 0 to 90% acetonitrile against 0.1% trifluoracetic acid/H₂O.

### LC-MS

LC-MS analysis was performed on a PE-Sciex API 100 or 150 mass spectrometer equipped with two Perkin Elmer Series 200 Micropumps, a Perkin Elmer Series 200 autosampler, a Applied Biosystems 785A UV detector and a Sedex 75 Evaporative Light scattering detector. A Waters Xterra 3.0 mm×50 mm 5μ C-18 silica column was eluted at 1.5 ml/min at room temperature. It was equilibrated with 5% acetonitrile/0.1% trifluoracetic acid/H₂O and eluted for 1.0 min with 5% acetonitrile/0.1% trifluoracetic acid/H₂O and then with a linear gradient to 90% acetonitrile/0.1% trifluoracetic acid/H₂O over 7 min. Detection was by UV detection at 214 nm and Evaporative light Scattering. A fraction of the column eluate was introduced into the ionspray interface of a PE-Sciex API 100 mass spectrometer. The mass range 300-2000 amu was scanned every 2 seconds during the run.

### Quantification of Protein

Protein concentrations were estimated by measuring absorbance at 280 nm using a NanoDrop ND-1000 UV-spectrofotometer.

### Enzymatic Peptide Mapping for Determination of Site(s) of Derivatization

Peptide mapping was performed using Asp-N digestion of the reduced and alkylated protein. First the protein was treated with DTT (Dithiothreitol) and iodoacetamide according to standard procedures. The alkylated product was purified using HPLC. Subsequently the alkylated purified product was digested overnight with endoprotease Asp-N (Boehringer) at an enzyme:substrate ratio of 1:100. The digest was HPLC separated using a C-18 column and standard trifluoracetic acid/acetonitrile buffer system. The resulting peptide map was compared to that of un-derivatized hGH and fractions with different retention times were collected and further analyzed using Maldi-tof mass spectrometry.

### SDS Page

SDS poly-acrylamide gel electrophoresis was performed using NuPAGE 4%-12% Bis-Tris gels (Invitrogen NP0321 BOX). The gels were silver stained (Invitrogen LC6100) or Coomassie stained (Invitrogen LC6065) and where relevant also stained for PEG with barium iodide as described by M. M. Kurfurst in Anal. Biochem. 200(2):244-248, 1992.

### Protein Chromatography

Protein chromatography was performed on an Akta Explorer chromatographic system and columns from GE Health Care. Anion exchange was done using a Q-Sepharose HP 26/10 column. Starting buffer was 20 mM triethanolamine buffer pH 8.5 and eluting buffer was starting buffer+0.2M NaCl. The compounds were typically eluted with a gradient of 0-75% eluting buffer over 15 column volumes. De-salting and buffer exchange was performed using a HiPrep 26/10 column.

### Example 1

### Preparation of SER-GHBP

The Ser-hGH analogue expression plasmid was created on the basis of pNNC13 (Zbasic2mt-D4K-hGH), which expresses the wild type hGH in fusion with Zbasic domain (mvdnkfnkerrrarreirhlpnlnreqrrapirslrddpsqsanllaeakklnraqapkyrggsddddksfptiplsrlfdnamlrahrl hqlafdtyqefeeayipkeqkysflqnpqtslcfsesiptpsnreetqqksnlellrisllliqswlepvqflrsvfanslvygasdsnvyd llkdleegiqtlmgrledgsprtgqifkqtyskfdtnshnddallknygllycfrkdmdkvetflrivqcrsvegscgf).

Additional Ser was inserted in front of Phe, the first amino acid of mature hGH, by QuikChange® XL Site-Directed Mutagenesis Kit from Stratagene with a pair of primes:

5′ end: pNNC13 Ser-F
5′-GGATCAGACGACGACGACAAAagcTTCCCAACCATTCCCTTATCC-3′
and
3′ end: pNNC13 Ser-R
5′-GGATAAGGGAATGGTTGGGAAgctTTTGTCGTCGTCGTCTGATCC-
3′.

E. coli BL21(DE3) was transformed by pET11a-Zbasic2mt-D4K-Ser-hGH. Single colony was inoculated into 100 ml LB media with 100 μg/ml Amp and grew at 37° C. When OD600 reached 0.6, the cell culture temperature was reduced to 30° C., and the cells were induced with 1 mM IPTG for 4 hours at 30 degree. The bacteria cells were harvested by centrifugation at 3000 g for 15 minutes (Eppendorf centrifuge 5810R). The cell pellet was re-suspended in cell lysis buffer (25 mM Na2HPO4 25 mM NaH2PO4 pH 7, 5 mM EDTA, 0.1% Triton X-100), and the cells were disrupted by cell disruption at 30 kpsi (Constant Cell Disruption Systems). The lysate was clarified by centrifugation at 10000 g for 30 minutes. The supernatant was saved and used for purification, while the pellet was discarded.

Zbasic2mt-D4K-Ser-hGH was purified on SP-Sepharose using a step gradient elution (buffer A: 25 mM Na2HPO4 25 mM NaH2PO4 pH 7; buffer B: 25 mM Na2HPO4 25 mM NaH2PO4 pH 7, 1 M NaCl). The protein was subsequently cleaved using Enteropeptidase for the release of Ser-hGH. Ser-hGH was further purified on a Butyl Sepharose 4FF column to separate the product from the Zbasic2mt-D4K domain and Enteropeptidase (buffer A: 100 mM Hepes pH 7.5; buffer B: 100 mM Hepes pH 7.5, 2 M NaCl, a linear gradient was used). The final product of Ser-hGH was buffer exchanged and lyophilized from 50 mM NH₄HCO₃, pH 7.8.

### Example 2

### Preparation of N-carbonyloxybenzyl-glutaminyl-glycyl-(4-amino-phenylalanine)[Z-Gln-Gly-(4-amino-Phe)-OH], Compound 2

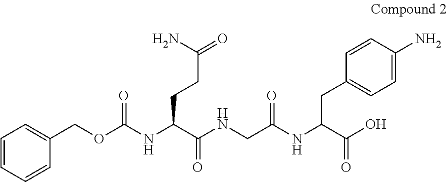

Attachment of the first amino acid to the resin: To a 2-chlorotrityl chloride resin (Pepchem, 2 g, 1.5 mmol/g) was added Fmoc-(4-Boc-amino-Phe)-OH (Fluka, 2.26 g, 4.5 mmol) dissolved in a mixture of DCM (16 ml) and diisopropylethylamine (780 μl). The slurry was stirred for 5 min followed by addition of diisopropylethylamine (1540 μl). Stirring was continued for a period of 1 h after which methanol (5 ml) was added, and stirring was continued for additional 15 min. The resin was drained and washed with dichloromethane (DCM) (6×30 ml) followed by N-methylpyrrolidone (NMP) (6×30 ml).

Removal of the Fmoc group: To the resin was added 20% piperidine in NMP (20 ml) and left reacting for 15 min. The resin was drained and again treated with 20% piperidine in NMP (20 ml) for 1 h. The resin was drained and washed with NMP (6×30 ml). Coupling Z-Gln-Gly-OH: To the resin was added a solution of Z-Gln-Gly-OH (Bachem, 1.52 g, 4.5 mmol) and hydroxybenzotriazole (HOBt, 0.61 g, 4.5 mmol) in NMP followed by diisopropylcarbodiimide (DIC) (700 μl, 4.5 mmol). After reaction overnight, the resin was drained and washed with NMP (6×30 ml), then with DCM (6×30 ml).

Cleavage from the solid support: The resin was drained to remove bulk DCM. It was treated with a mixture of trifluoroacetic acid (TFA) (12.6 ml), water (0.6 ml), DCM (5.8 ml) and triisopropylsilane (0.8 ml). After reaction for 1 h, the resin was filtered slowly within 15 min into diethylether (100 ml) which was stirred for an additional 30 min. The resuling precipitate was recovered by centrifugation and washed 3 times with diethylether. The solid was dried overnight in vacuo. The product was pure and homogenous according to ¹H-NMR and LC-MS.

### Example 3

### Transamidation of hGH with Z-Gln-Gly-(4-amino-Phe)-OH (compound 2) to obtain Z-Gln(hGH)-Gly-(4-amino-Phe)-OH, Compound 3

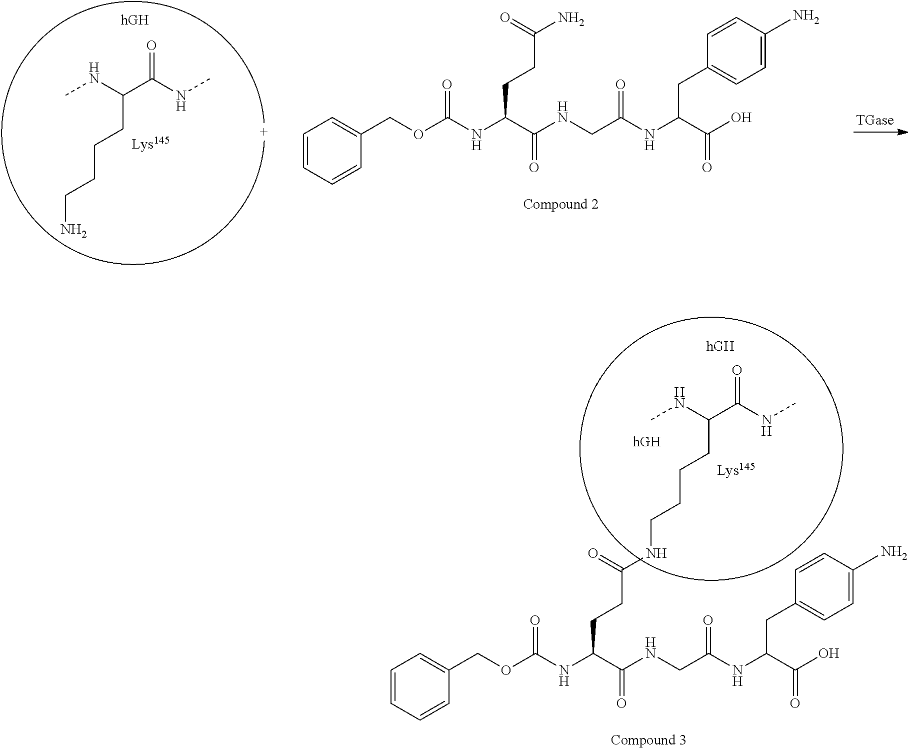

Three solutions are prepared: 1) hGH (40 mg, 1.8 μmol) dissolved in 1 ml 20 nM triethanolamine buffer pH 8.5; 2) Z-Gln-Gly-OH (202 mg, 412 μmol) dissolved in 2 ml 20 nM triethanolamine buffer pH 8.5, pH adjusted to 8.15 using 10% triethanolamine solution (2.4 ml); 3) Transglutaminase (1% in solid mixture with maltodextrin, 36 mg, 9 nmol) was dissolved in 1 ml 20 nM triethanolamine buffer pH 8.5.

Solutions 1 and 2 with mixture and 111 μl of solution 3 was added. pH was 8.2 and volume 5.5 ml. The reaction was monitored by CE. After 5 h reaction at r.t., Analysis by CE showed the presence of a new product with an increased migration time, showing about 70% conversion to the transamidated product. To the reaction mixture was added 10 mM aqueous N-ethylmaleimide (300 μl) and it was stored at 5 ° C. overnight. The mixture was loaded to a 15 ml HiPrep column (GE Healthcare) and eluted using triethanolamine buffer pH 8.5 to remove low molecular weight substances and salt. Relevant fractions were pooled and the recovery was 36.6 mg protein based on UV absorption measurements.

### Example 4

### Oxidation of Ser-GHBP to Glyoxalyl-GHBP

Ser-GHBP is oxidated to glyoxalyl-GHBP as described in Example 6 below.

### Example 5

Reductive Amination of Glyoxalyl-GHBP with Compound 3 from Example 3

Glyoxalyl-hGHBP is subjected to reductive amination with compound 3 from Example 3 analogous to what is described in section (E) of Example 6 to give the GH-GHBP conjugate.

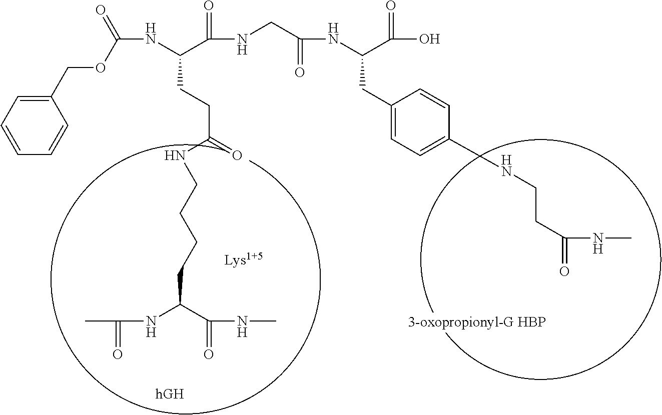

### Example 6

### (A) Oxidation of Ser-hGH (I) to glyoxalyl-hGH (II)

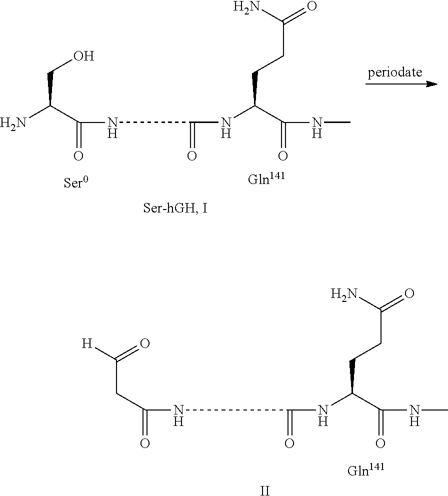

The following solutions were prepared:

Buffer A: Triethanolamine (119 mg, 0.8 mmol) was dissolved in water (40 ml). pH was adjusted to 8.5

Buffer B: 3-methylthiopropanol (725 mg, 7.1 mmol) was dissolved in Buffer A (10 ml).

Periodate: NaIO₄ (48.1 mg, 0.225 mmol) was dissolved in water (1.0 ml).

4-aminobenzoic acid: 10 mg (73 μmol) 4-aminobenzoic acid (Mwt.: 137) was dissolved in 2.5 ml 50% acetic acid, 50% Buffer A

Ser-hGH (50 mg, 2.3 μmol) was dissolved in cold buffer A (5.0 ml), and added 1.0 ml Buffer B. The periodate solution (0.5 ml) was added. After standing at 4° C. (refrigegator) for 20 min the mixture was transferred to a dialysis tube (Amicon Ultra-15 device; cut-off 10.000), and dialyzed four times with buffer A

The residue was concentrated to a volume of 2.5 ml.

### (B) Reductive Amination of Glyoxalyl-hGH II with 4-aminobenzoic acid

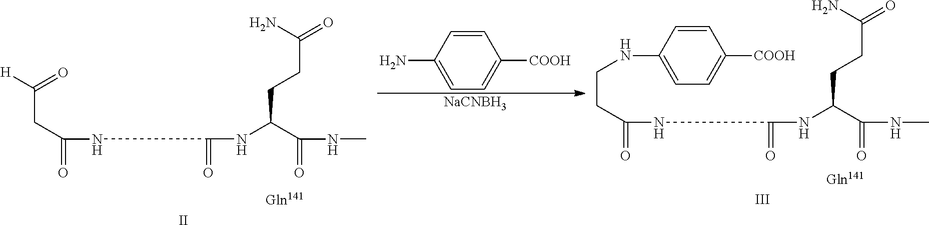

The solution of oxidized Ser-hGH was added immediately after its preparation to the solution of 4-aminobenzoic acid, and the resulting mixture was slowly rotated at 4° C. After 1 h NaCNBH₃ (100 μl of a solution of 20 mg NaCNBH₃ and 15 μl AcOH in 0.5 ml water) was added. The mixture was kept at 4° C. in the dark.

After 42 h the mixture was diluted 10 times with Buffer A, and the product III was purified by ion exchange chromatography.

### (C) Transamination of III with 1,3-diamino-2-propanol

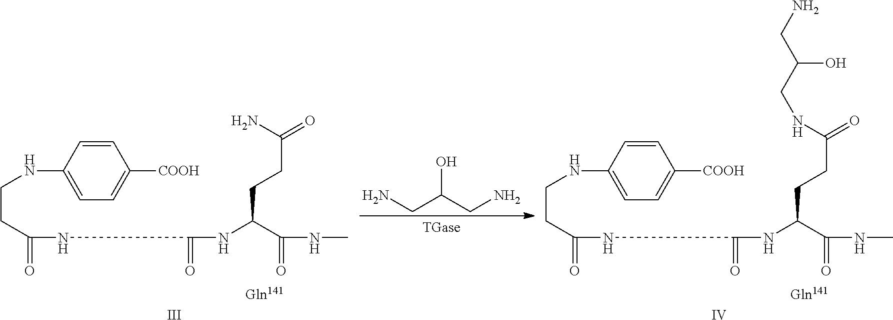

The following solutions were prepared:

Buffer A: Triethanolamine (119 mg, 0.8 mmol) was dissolved in water (40 ml). pH was adjusted to 8.5

Nucleophile solution: 1,3-diaminopropanol (90 mg, 1.0 mmol) was dissolved in Buffer A (300 μl). The pH was adjusted to 8.5 with concentrated hydrochloride acid, and the volume was adjusted to 600 μl with Buffer A.

Enzyme solution: 25 mg TGase was dissolved in 200 μl Buffer A

The purified product III was concentrated to 1.7 ml by ultrafiltration. 1.0 ml Ethylene glycol (30%) was added to the solution along with the nucleophile solution. Finally the enzyme solution was added and the total volume was adjusted to 3.5 ml with Buffer A.

The reaction was left at room temperature for 4-6 hours.

The reaction solution was diluted 10 times with buffer A and the product IV was purified by ion exchange chromatography.

### (D) Oxidation of IV

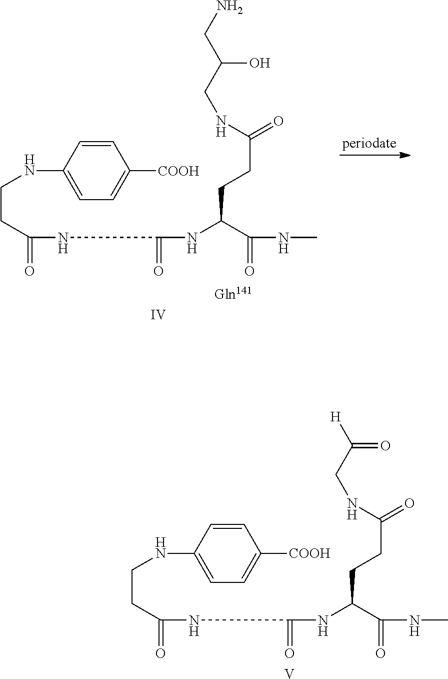

The following solutions were prepared:

Buffer B: 3-methylthiopropanol (725 mg, 7.1 mmol) was dissolved in Buffer A (10 ml).

Buffer C: HEPES (5.96 g) was dissolved in water (1.0 l). pH was adjusted to 7.0

Periodate: NaIO₄ (48.1 mg, 0.225 mmol) was dissolved in water (1.0 ml).

To a solution of IV (10 mg, 0.5 μmol) 0.2 ml Buffer B was added and then the periodate solution (0.03 ml). After 20 min's of cold incubation the mixture was dialyzed 4 times with buffer C. The residue was concentrated to 1 ml.

### (E) Reductive Amination of V with GHBP

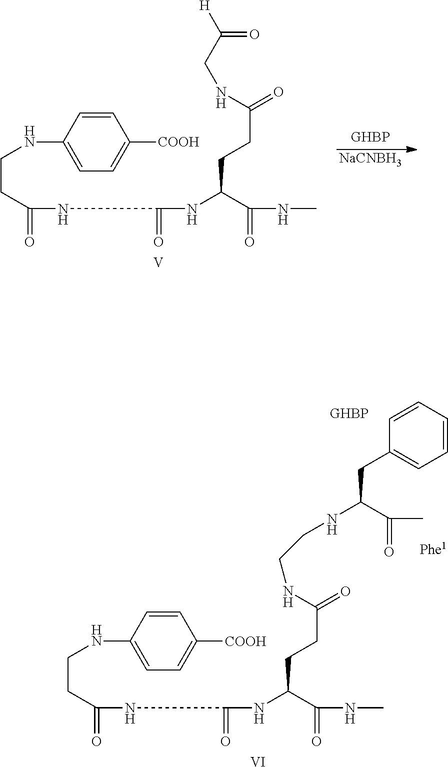

GHBP is obtained using similar methods as those described by M. Sundstrom et al. in J. Biol. Chem. 271 (50):32197-32203, 1996 with the exception that the GHBP after the final ion exchange chromatography step is dialyzed with a 25 mM HEPES buffer pH 7.0 and concentrated to a final concentration of 5 mg/ml.

The final solution from D. (1 ml, 10 mg, 0.45 μmol V) is mixed with a GHBP solution (2 ml, 10 mg 0.3 μmol) in a 25 mM HEPES buffer pH 7.0 and the resulting mixture is slowly rotated at room temperature.

After 1 h NaCNBH₃ (100 μl of a solution of 20 mg NaCNBH₃ in 0.5 ml water) is added portionwise. The mixture is kept at room temperature in the dark for 18-24 hours.

The mixture is diluted with 1M tris solution to a final concentration of 50 mM pH 7.5 and applied to an ion exchange column and the product VI is obtained by elution of the column with a gradient of NaCl₂

### Example 7

### BAF-3GHR Assay to Determine Growth Hormone Activity

BAF-3 cells (a murine pro-B lymphoid cell line derived from the bone marrow) are originally IL-3 dependent for growth and survival. IL-3 activates JAK-2 and STAT which are the same mediators GH is activating upon stimulation. After transfection of the hGH receptor the cell line is transformed into a growth hormone-dependent cell line. This clone can be used to evaluate the effect of different growth hormone samples on the survival of the BAF-3GHR.

The BAF-3GHR cells are grown in starvation medium (culture medium without growth hormone) for 24 h at 37° C., 5% CO₂.

The cells are washed and resuspended in starvation medium and seeded in plates. 10 μl of growth hormone compound or hGH in different concentrations or control is added to the cells, and the plates are incubated for 68 h at 37° C., 5% CO₂.

AlamarBlue® is added to each well and the cells are incubated for further 4 h. AlamarBlue® is a redox indicator, which is reduced by reactions innate to cellular metabolism and, therefore, provides an indirect measure of viable cell number.

The metabolic activity of the cells was measured in a fluorescence plate reader. The absorbance in the samples is expressed in % of cells not stimulated with growth hormone compound or control, and from the concentration-response curves the activity (amount of a compound that stimulates the cells with 50%, EC₅₀) could be calculated.

1. A compound having the formula:
A-B-C,
wherein A is a radical of a growth hormone compound;
B is a linking and spacing bivalent residue;
C is a radical of a growth hormone binding protein compound; and
- is a covalent bond,
wherein the linkages between A and B and/or the linkage between B and C are not provided by recombinant expression of a nucleic acid comprising a nucleic acid sequence encoding a fusion protein having the structure A-B-C.

2. A compound having the formula:
A-B-C,
wherein A is a radical of a growth hormone compound;
B is a linking and spacing bivalent residue;
C is a radical of a growth hormone binding protein compound; and
- is a covalent bond,
wherein B is not a pure peptide chain.

3. A compound according to claim 1, wherein at least one of the covalent bonds A-C or B-C is established by an enzyme.

4. A compound according to claim 1, wherein B comprises at least 10 covalent bonds.

5. A compound according to claim 1, wherein B comprises a PEG unit and wherein B optionally comprises the structure; 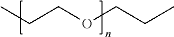
wherein n is an integer larger than 1, and the molecular weight of the structure is between around 100 Da to around 1,000,000 kDa.

6. A compound according to claim 1, wherein B is selected from the group consisting of:

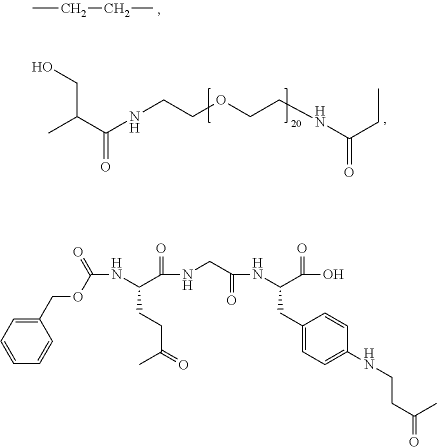

7. A compound according to claim 3 of the formula 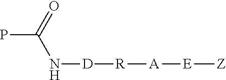
wherein P—C(O)—NH— represents the growth hormone compound radical obtained by removing a hydrogen from —NH₂ in the side chain of Gln;
D represents a bond or oxygen;
R represents a linker or a bond;
E represents a linker or a bond;
A represents an oxime, hydrazone, phenylhydrazone, semicarbazone, triazole or isooxazolidine moiety; and
Z is a radical of a growth hormone binding protein compound.

8. A compound according to claim 3 of the formula (I) 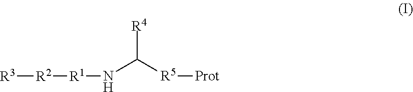
wherein
Prot represents a radical of the growth hormone compound, wherein said radical is formally generated by the formal removal of a hydrogen atom from an amino group (—NH₂) of said growth hormone compound,
R¹ represents arylene or a heteroarylene, optionally substituted with a C₁₆-alkyl, halogen, cyano, nitro, hydroxyl, carboxyl, or aryl group;
R² represents a bond or a linker, wherein said linker comprises a diradical selected from the group consisting of C(═O)NH, NH, O, S, OP(O)(OH)O, OC(═O)NH, NHC(═O)NH, (CH₂)₁₁₀, O(CH₂)₃NHC(═O), C(═O)NH(CH₂)₂₃₀, (CH₂)₁₃₀C(═O)NH(CH₂)₂₃₀, (CH₂)₀₃₀C(═O)NH(CH₂CH₂O)₁₁₀(CH₂)₁₅C(═O), C(═O)NH[(CH₂CH₂O)₁₁₀(CH₂)₁₅C(═O)]₁₅NH(CH₂)₂₃₀, C(═O), (CH₂)₁₃₀—NHC(═O), (CH₂)₁₃₀C(═O), NHC(═O)NH(CH₂)₂₃₀, (CH₂)₁₃₀—NHC(═O)NH(CH₂)₂₃₀, (CH₂)₀₃₀C(═O)NH(CH₂)₂₃₀NHC(═O)(CH₂)₀₃₀, (CH₂)₀₃₀C(═O)NH(CH₂CH₂O)₁₃₀CH₂CH₂NHC(═O)(CH₂)₀₃₀, or NH(CH₂)₂₃₀, 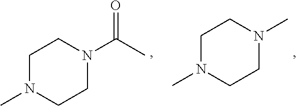
and combinations thereof, and
R³ represents the radical of the growth hormone binding protein compound;
R⁴ represents hydrogen or C₁₆-alkyl; and
R⁵ represents —CH₂— or —C(═O)—.

9. A pharmaceutical composition comprising a compound according to claim 1 and a pharmaceutically acceptable carrier.

10. A method of treating a disease state in a mammal that will benefit from increase in the activity of growth hormone comprising administering to the mammal an effective amount of a pharmaceutical composition according to claim 9.

11. A method for preparing a compound according to claim 1, said method comprising
(a) enzymatic derivitization of the growth hormone compound, and
(b) conjugating the enzymatically derivatized growth hormone compound from step (a) to the growth hormone binding protein compound.

12. A method for preparing a compound according to claim 3, which method comprises
i) contacting the growth hormone compound with a first compound having the following formula: R⁷GlnGlyR⁸, wherein R⁷ and R⁸ are groups suitable for further modification, in the presence of a transglutaminase, and
ii) reacting in one or more steps the product of step i) with a second compound comprising one or more functional groups, wherein said functional group(s) do not react with functional groups accessible in the amino acid residues constituting said peptide, and wherein said functional group(s) in said second compound is capable of reacting with R⁷ and/or R⁸, so that a covalent bond between the product of step i) and said second compound is formed, and wherein said second compound comprises the radical of the growth hormone binding protein compound.

13. A method for preparing a compound according to claim 7, wherein said method comprising the steps of
i) reacting in one or more steps the growth hormone compound with a first compound comprising one or more functional groups or latent functional groups, which are not accessible in any of the amino acids residues constituting said growth hormone compound, in the presence of transglutaminase capable of catalysing the incorporation of said first compound into said growth hormone compound to form a functionalised growth hormone compound; and
ii) optionally activateing the latent functional group; and
iii) reacting in one or more steps said functionalised growth hormone compound with a second compound comprising one or more functional groups, wherein said functional group(s) do not react with functional groups accessible in the amino acid residues constituting said peptide, and wherein said functional group(s) in said second compound is capable of reacting with said functional group(s) in said first compound so that a covalent bond between said functionalised peptide and said second compound is formed, and wherein said second compound comprises the radical of the growth hormone binding protein compound.

14. A method for preparing a compound according to claim 8, said method comprising the steps of (a) treating an aldehyde or ketone derived from the growth hormone compound with a property-modifying group-derived aniline or heteroarylamine to yield an imine or a hemiaminal, wherein said property-modifying group comprises a growth hormone binding protein or fragment thereof,
(b) treating this imine or hemiaminal with a suitable reducing agent, such as NaCNBH₃, to yield a secondary amine.

15. A compound of formula (III)
R³—R²—R¹—NH₂ (III)
wherein
R¹ represents arylene or a heteroarylene, optionally substituted with a C₁₆-alkyl, halogen, cyano, nitro, hydroxyl, carboxyl, or aryl group;
R² represents a bond or a linker, wherein said linker comprises a diradical selected from the group consisting of C(═O)NH, NH, O, S, OP(O)(OH)O, OC(═O)NH, NHC(═O)NH, (CH₂)₁₁₀, O(CH₂)NHC(═O), C(═O)NH(CH₂)₂₃₀, (CH₂)₁₃₀C(═O)NH(CH₂)₂₃₀, (CH₂)₀₃₀C(═O)NH(CH₂CHO)₁₁₀(CH₂)₁₅C(═O), C(═O)NH[(CH₂CH₂O)₁₁₀(CH₂)₁₅C(═O)]₁₅NH(CH₂)₂₃₀, C(═O), (CH₂)₁₃₀—NHC(═O), (CH₂)₁₃₀C(═O), NHC(═O)NH(CH₂)₂₃₀, (CH₂)₁₃₀—NHC(═O)NH(CH₂)₂₃₀, (CH₂)₀₃₀C(═O)NH(CH₂)₂₃₀NHC(═O)(CH₂)₀₃₀, (CH₂)₀₃₀C(═O)NH(CH₂CH₂O)₁₃₀CH₂CH₂NHC(═O)(CH₂)₀₃₀, or NH(CH₂)₂₃₀, 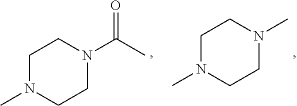
and combinations thereof; and
R³ represents the radical of the growth hormone binding protein compound.

16. A compound according to claim 2, wherein at least one of the covalent bonds A-C or B-C is established by an enzyme.

17. A compound according to claim 2, wherein B comprises at least 10 covalent bonds.

18. A compound according to claim 2, wherein B comprises a PEG unit and wherein B optionally comprises the structure; 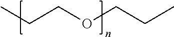
wherein n is an integer larger than 1, and the molecular weight of the structure is between around 100 Da to around 1,000,000 kDa.

19. A compound according to claim 2, wherein B is selected from the group consisting of:

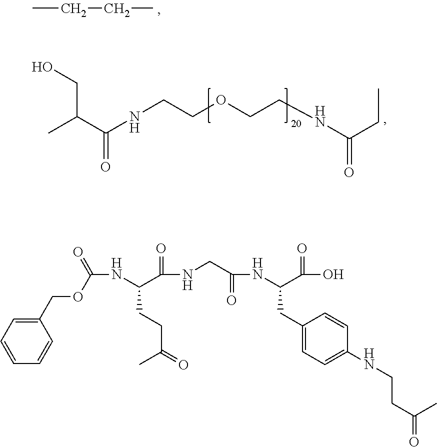

20. A compound according to claim 16 of the formula 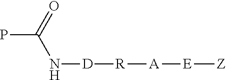
wherein P—C(O)—NH— represents the growth hormone compound radical obtained by removing a hydrogen from —NH₂ in the side chain of Gln;
D represents a bond or oxygen;
R represents a linker or a bond;
E represents a linker or a bond;
A represents an oxime, hydrazone, phenylhydrazone, semicarbazone, triazole or isooxazolidine moiety; and
Z is a radical of a growth hormone binding protein compound.

21. A compound according to claim 16 of the formula (I) 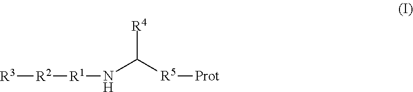
wherein
Prot represents a radical of the growth hormone compound, wherein said radical is formally generated by the formal removal of a hydrogen atom from an amino group (—NH₂) of said growth hormone compound,
R¹ represents arylene or a heteroarylene, optionally substituted with a C₁₆-alkyl, halogen, cyano, nitro, hydroxyl, carboxyl, or aryl group;
R² represents a bond or a linker, wherein said linker comprises a diradical selected from the group consisting of C(═O)NH, NH, O, S, OP(O)(OH)O, OC(═O)NH, NHC(═O)NH, (CH₂)₁₁₀, O(CH₂)₃NHC(═O), C(═O)NH(CH₂)₂₃₀, (CH₂)₁₃₀C(═O)NH(CH₂)₂₃₀, (CH₂)₀₃₀C(═O)NH(CH₂CH₂O)₁₁₀(CH₂)₁₅C(═O), C(═O)NH[(CH₂CH₂O)₁₁₀(CH₂)₁₅C(═O)]₁₅NH(CH₂)₂₃₀, C(═O), (CH₂)₁₃₀—NHC(═O), (CH₂)₁₃₀C(═O), NHC(═O)NH(CH₂)₂₃₀, (CH₂)₁₃₀—NHC(═O)NH(CH₂)₂₃₀, (CH₂)₀₃₀C(═O)NH(CH₂)₂₃₀NHC(═O)(CH₂)₀₃₀, (CH₂)₀₃₀C(═O)NH(CH₂CH₂O)₁₃₀CH₂CH₂NHC(═O)(CH₂)₀₃₀, or NH(CH₂)₂₃₀, 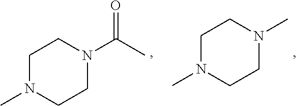
and combinations thereof;
R³ represents the radical of the growth hormone binding protein compound;
R⁴ represents hydrogen or C₁₆-alkyl; and
R⁵ represents —CH₂— or —C(═O)—.

22. A pharmaceutical composition comprising a compound according to claim 2 and a pharmaceutically acceptable carrier.

23. A method of treating a disease state in a mammal that will benefit from increase in the activity of growth hormone comprising administering to the mammal an effective amount of a pharmaceutical composition according to claim 22.

24. A method for preparing a compound according to claim 2, said method comprising
(a) enzymatic derivitization of the growth hormone compound, and
(b) conjugating the enzymatically derivatized growth hormone compound from step (a) to the growth hormone binding protein compound.

25. A method for preparing a compound according to claim 16, which method comprises
i) contacting the growth hormone compound with a first compound having the following formula: R⁷GlnGlyR⁸, wherein R⁷ and R⁸ are groups suitable for further modification, in the presence of a transglutaminase, and
ii) reacting in one or more steps the product of step i) with a second compound comprising one or more functional groups, wherein said functional group(s) do not react with functional groups accessible in the amino acid residues constituting said peptide, and wherein said functional group(s) in said second compound is capable of reacting with R⁷ and/or R⁸, so that a covalent bond between the product of step i) and said second compound is formed, and wherein said second compound comprises the radical of the growth hormone binding protein compound.

26. A method for preparing a compound according to claim 20, wherein said method comprising the steps of
i) reacting in one or more steps the growth hormone compound with a first compound comprising one or more functional groups or latent functional groups, which are not accessible in any of the amino acids residues constituting said growth hormone compound, in the presence of transglutaminase capable of catalysing the incorporation of said first compound into said growth hormone compound to form a functionalised growth hormone compound; and
ii) optionally activating the latent functional group; and
iii) reacting in one or more steps said functionalised growth hormone compound with a second compound comprising one or more functional groups, wherein said functional group(s) do not react with functional groups accessible in the amino acid residues constituting said peptide, and wherein said functional group(s) in said second compound is capable of reacting with said functional group(s) in said first compound so that a covalent bond between said functionalised peptide and said second compound is formed, and wherein said second compound comprises the radical of the growth hormone binding protein compound.

27. A method for preparing a compound according to claim 21, said method comprising the steps of
(a) treating an aldehyde or ketone derived from the growth hormone compound with a property-modifying group-derived aniline or heteroarylamine to yield an imine or a hemiaminal, wherein said property-modifying group comprises a growth hormone binding protein or fragment thereof,
(b) treating this imine or hemiaminal with a suitable reducing agent, such as NaCNBH₃, to yield a secondary amine.
